(19) United States
(12) Patent Application Publication
(10) Pub. No.: US 2007/0168043 A1
(43) Pub. Date: Jul. 19, 2007

(54) Percutaneous cervical disc reconstruction

(75) Inventor: Bret A. Ferree, Cincinnati, OH (US)

Correspondence Address: O'MELVENY & MYERS LLP, 610 NEWPORT CENTER DRIVE, 17TH FLOOR, NEWPORT BEACH, CA, 92660, US

(73) Assignee: Anova Corporation

(21) Appl. No.: 11/652,717

(22) Filed: Jan. 12, 2007

Related U.S. Application Data
(60) Provisional application No. 60/759,151, filed on Jan. 13, 2006.

(51) Int. Cl. A61F 2/44

(52) U.S. Cl. 623/17.16; 623/908; 623/17.11

(57) ABSTRACT

Methods and devices for fixing a defect in a vertebral disc of a patient. One method includes inserting a guide wire into a disc using an anterior approach to create a path. At least a portion of the herniated nucleus pulposus is removed. A disc reconstruction device is then advanced into the disc and fastened to the disc or at least one of the surrounding vertebrae. Alternatively, a passage could be formed in the cranial or caudal vertebra using an angled anterior approach that terminates in a region of the disc containing the herniated nucleus pulposus. At least a portion of the herniated nucleus pulposus is removed. A disc reconstruction device that has a bone in-growth component and a soft tissue in-growth component is then implanted in the passage. Devices having bone and soft tissue in-growth components and having a flexible spine with a plurality of ribs are also described.

### RELATED APPLICATIONS

This application claims priority to U.S. Provisional Application Ser. No. 60/759,151, filed Jan. 13, 2006, entitled “Percutaneous Cervical Disc Reconstruction,” which is hereby incorporated by reference in its entirety.

### BACKGROUND OF THE INVENTION

Spondylosis (spinal osteoarthritis) is a common degenerative disorder that is most likely caused by age-related changes in the discs. It can affect the cervical, thoracic, and/or lumbar regions of the spine and can result in loss of normal spinal structure and function. Structural alterations to the disc, e.g., weakening of the anulus fibrosus and decreased water content in the nucleus pulposus, may result in decreased disc height and increase the risk for disc herniation. Clinically, this can result in severe pain in the neck, shoulder, and upper limbs. Additionally, osteophytes (e.g., bone spurs) may form adjacent to the vertebral end plates. When an osteophyte causes nerve root compression, a patient may experience weakness in an arm or other extremity. When bone spurs form at the front of the cervical spine, a patient may experience difficulty in swallowing (dysphagia).

Anterior Cervical Discectomy and Fusion (ACD&F) was first described in the late 1950s. The procedure involves removing one or more discs and placing bone in the disc space or spaces. Bone tissue from the vertebra above and the vertebra below the bone graft grow into the graft, thus fusing the vertebrae together. Anterior cervical discectomy and fusion is the most common surgical procedure performed on the cervical spine. The procedure is used to treat herniation of the nucleus pulposus (HNP), spondylosis, spinal stenosis, infection, and tumors.

Spinal fusion eliminates movement at the operative level. The abnormal kinematics cause accelerated degeneration of the discs above and below the fused level. Up to twenty percent of patients require surgery to treat degeneration of the discs adjacent to the fusion. The medical community would welcome alternative surgical treatments for degenerative conditions of the cervical spine.

### SUMMARY OF THE INVENTION

Technology has advanced substantially over the last fifty years. The invention applies the advancements in medical technology to provide a non-fusion procedure to treat degenerative conditions of the cervical spine, such as spondylosis. The invention generally involves the use of image guidance to remove herniated nucleus pulposus and osteophytes (bone spurs) of the cervical spine. Imagine guidance includes the use of CT, MRI, fluoroscopy, and/or navigation to direct instruments into the disc. Image guidance enables the interventionalist to preserve the discs, only the portion of the patient's disc that is causing the patient's symptoms is removed.

The invention also includes reconstructing the disc after removal of the pathological portion of the disc. The reconstruction device is made of material, such as polyester mesh, that promotes tissue in-growth. The procedure may be performed under local anesthesia through an incision in the skin of one centimeter in length or less. Conscious patients can notify the physician if they experience new arm or leg symptoms. New symptoms in the extremities suggest one of the instruments may be applying excessive pressure on a nerve. The instruments could deliver electrical impulses to help stimulate the nerves before the instrument contacts the nerve. Conscious patients may also notify the physician if their pre-operative symptoms resolve. Improvement of the patient's symptoms suggests the HNP has been removed.

Although the invention is preferably used in the cervical spine, it may be also be used in the thoracic and lumbar portions of the spine. The invention may be used to insert catheters near the herniated nucleus pulposus. The catheters may be connected to an internal or external reservoir. Local anesthetics, anti-inflammatory agents, platelets or other materials may be delivered to the injured disc through the catheter.

In one aspect of the invention, a method of repairing a disc having a herniated nucleus pulposus is described. The disc is situated between a cranial and caudal vertebra. A guide wire is inserted into the disc using an anterior lateral approach to create a path. At least a portion of the nucleus pulposus is then removed. A suture is attached to at least one of the cranial or caudal vertebra and a disc reconstruction device is advanced along the path into the disc. The disc reconstruction device is then fastened to at least one of the cranial or caudal vertebra with the suture.

The disc reconstruction device may be made from a porous mesh or a biologic material such as allograft tissue, autograft tissue, xenograft tissue, tendons, fascia, demineralized bone matrix, intestinal sub-mucosa, and dermis. The disc reconstruction device may have a variety of different shapes including a generally cylindrical shape, a plurality of elongate extensions, a tubular shape with a lumen therethrough.

Additionally, a tissue dilator may be used to enlarge the passage in the disc along the path. Various instruments could then be used to remove at least a portion of the nucleus pulposus. These include a grasping tool, a pinching tool, and an elongate member having a corkscrew located at its distal end.

In yet another embodiment of the invention, another method for repairing a disc having a herniated nucleus pulposus is described. The disc is situated between a cranial and caudal vertebra. A passage is formed in the cranial or caudal vertebra that extends into the disc using an angled anterior approach that enters the cranial or caudal vertebra and terminates at a posterior region of the disc that includes the herniated nucleus pulposus. A portion of the herniated nucleus pulposus is then removed. A disc reconstruction device is implanted into the passage that has a bone in-growth component and a tissue in-growth component. The disc reconstruction device is implanted such that the bone in-growth component is located in the cranial or caudal vertebra and the tissue in-growth component is located in the disc.

The components of the disc reconstruction device will be made from material that promote bone or tissue in-growth. The bone in-growth component could be made from bone, titanium, ceramic, or tantalum. Additionally, the bone in-growth component may have external threads that are adapted to frictionally engage either the cranial or caudal vertebra. The tissue in-growth component could be made from a porous mesh or a biologic material such as allograft tissue, autograft tissue, xenograft tissue, tendons, fascia, demineralized bone matrix, intestinal sub-mucosa, and dermis.

In yet another embodiment of the invention, a method for removing a bone spur from a vertebra is described. A guide wire is inserted into a disc located adjacent to the vertebra using an anterior lateral approach to create a path. A cutting tool is then advanced along the path. At least a portion of the bone spur is then removed from the vertebra using the cutting tool. A disc reconstruction device is then advanced along the path into the disc. The disc reconstruction device is then fastened to at least one of the disc or the vertebra.

In alternative methods, the method described above may include the step of forming an enlarged passage in the disc along the path made by the guide wire. The disc reconstruction device may be passed over the guide wire to the region of interest. Additionally, the disc reconstruction device may be fastened to the disc with an anchor. The anchor may have first and second transverse ends, Alternatively, the device may be fastened to at least one of the cranial or caudal vertebra adjacent the disc with a suture anchor.

In yet another embodiment of the invention, a medical device for implantation into a spine of a patient is described. The medical device has a soft tissue in-growth component and a hard tissue in-growth component. As stated previously, the hard tissue in-growth component could be made from bone, titanium, ceramic, or tantalum. Additionally, the hard tissue in-growth component may have external threads that are adapted to frictionally engage either the cranial or caudal vertebra. The soft tissue in-growth component could be made from a porous mesh or a biologic material such as allograft tissue, autograft tissue, xenograft tissue, tendons, fascia, demineralized bone matrix, intestinal sub-mucosa, and dermis. Furthermore, in another embodiment, the device may further include a mesh sleeve having a closed distal end, wherein the soft issue in-growth component is located distal of the hard tissue in-growth component within the sleeve.

In yet another embodiment of the invention, an alternative medical device for implantation into a spine of a patient is described. The device includes a mesh sleeve having a chamber and a closed distal end. The device further includes a soft tissue in-growth component and a hard tissue in-growth component located within the mesh chamber of the mesh sleeve. The soft tissue component is located distal of the hard tissue in-growth component in the sleeve chamber. The mesh sleeve may further include extensions that extend beyond the hard tissue component. These extensions can be used to fasten the device to the surrounding vertebra or disc. Additionally, the mesh sleeve may also include a flap that can cover an open proximal end of the sleeve, thereby trapping or securing the soft and hard tissue in-growth components in the chamber.

In yet another embodiment of the invention, an alternative medical device for implantation into a spine of a patient is described. The medical device includes a soft tissue in-growth component comprising a porous mesh and an elongate member extending proximally from the soft tissue in-growth component. It also includes an elongate threaded member. In one embodiment, the elongate threaded member is adapted to frictionally engage the elongate member at a position along a circumference of the elongate threaded member. Alternatively, the elongate threaded member may have a lumen adapted to receive the elongate member. In this embodiment, the device includes a fastener releasably attached to a distal end of the elongate member.

In the above embodiments, the soft tissue in-growth component may have many different configurations. The soft tissue in-growth component may be a coiled piece of porous mesh, have a plurality of appendages adapted to radially expand, or have an expandable metal or plastic frame. The soft tissue in-growth component having the expandable appendages or expandable frame may further be covered by a porous mesh to help facilitate tissue in-growth. The porous mesh may be composed of polypropylene, polyester, and/or ePTFE. Additionally, the elongate member may be a suture.

In yet another embodiment of the invention, an alternative medical device for implantation into a spine of a patient is described. The medical device includes an expandable implant composed of an elastic or shape-memory material and an elongate member extending proximally from the expandable implant. It also includes an elongate threaded member. In one embodiment, the elongate threaded member is adapted to frictionally engage the elongate member at a position along a circumference of the elongate threaded member. Alternatively, the elongate threaded member may have a lumen adapted to receive the elongate member. In this embodiment, the device includes a fastener releasably attached to a distal end of the elongate member.

In the above embodiments, the expandable implant may have many different configurations. The expandable implant may have a plurality of appendages adapted to radially expand or have an expandable metal or plastic frame. Furthermore, the expandable implant may be covered by a porous mesh to help facilitate tissue in-growth. The porous mesh may be composed of polypropylene, polyester, and/or ePTFE. Additionally, the elongate member may be a suture.

In another embodiment, the invention includes methods for repairing a disc having a herniated nucleus pulposus using the medical devices described above. The disc is situated between a cranial and caudal vertebrae. A disc reconstruction device having an expandable implant and an elongate member extending from the expandable implant is placed in the passage. An elongate threaded member is then placed in the passage in the vertebra such that the elongate threaded member at least partially fills the passage.

The elongate threaded member described above has alternative configurations. In one embodiment, the elongate threaded member is placed adjacent to the elongate member in the passage such that the elongate threaded member frictionally engages the elongate member along a circumference of the elongate threaded member. In another embodiment, the elongate threaded member has a lumen and the elongate member is received within the lumen of the elongate threaded member. A fastener can then be secured on the elongate member at a proximal end of the elongate threaded member.

In another embodiment, the invention includes a medical device for implantation into a spine of a patient. The device includes an elongate tubular structure having a proximal end, a tapered distal end, and a lumen therebetween. Furthermore, the elongate tubular structure may be composed of a porous mesh made from a shape memory material or metal, such as nitinol. Additionally, the device may have an open proximal end.

In use, the device described above can be used to repair a disc having a herniated nucleus pulposus, wherein the disc is situated between a cranial and caudal vertebra. A passage is formed in the cranial vertebra using an angled anterior approach, e.g., an angled anterior lateral approach, that enters the cranial vertebra and terminates at a posterior region of the disc that includes the herniated nucleus pulposus. A portion of the herniated nucleus pulposus is removed. An elongate tubular structure is then placed in the passage, wherein the elongate tubular structure has a proximal end, a tapered distal end, and a lumen therebetween. The elongate tubular structure is then expanded to frictionally engage the vertebral surface of the passage.

The elongate tubular structure may be expanded by multiple methods. In one method, the elongate tubular structure is made from a shape memory metal and expands as a result of a temperature change. In another embodiment, the elongate tubular structure expands as a result of inflating a balloon or other inflatable member within the lumen of the elongate tubular structure. Furthermore, the elongate tubular structure may be made of a porous mesh.

In yet another embodiment, the invention includes an alternative medical device for implantation into a spine of a patient. The medical device includes a generally rectangular member having first and second longitudinal edges and a plurality of ribs extending from the first and second longitudinal edges. The generally rectangular member and plurality of ribs are composed of an elastic or shape-memory material and the device is expandable from a contracted state to an expanded state.

In the above-described device, the contracted and expanded states can be described in various ways. For instance, a distance between a free end of a rib on the first longitudinal edge and a free end of a corresponding rib on the second longitudinal edge is smaller in the contracted state than in the expanded state. Alternatively, the generally rectangular member and the plurality of ribs define a volume and the defined volume is smaller in the contracted state than in the expanded state.

The plurality of ribs may further have a projection, such as a tine, tooth, or barb, on an outer surface to aid in frictionally engaging the vertebral surface. The plurality of ribs may be composed of nitinol or another shape memory material. Additionally, where each of the generally rectangular member and the plurality of ribs have a thickness, the thickness of the generally rectangular member is larger than the thickness of the plurality of ribs.

In use, the device described above can be used to repair a disc having a herniated nucleus pulposus, wherein the disc is situated between a cranial and caudal vertebra. A passage is formed in the cranial vertebra that extends into the disc using an angled anterior approach, such as an anterior lateral approach, that enters the cranial vertebra and terminates at a posterior region of the disc that includes the herniated nucleus pulposus. A device having a generally rectangular member having first and second longitudinal edges and a plurality of ribs extending from the first and second longitudinal edges is placed into the passage. The device is expanded to frictionally engage the vertebral surface of the passage.

The device can expand using various techniques. Where the device is composed of a shape memory material, the device can automatically expand as a result of a temperature change after it is located in the passage. Where the device is composed of an elastic material, the device can expand as a result of expanding or inflating a balloon located between the plurality of ribs such that the ribs are pushed outward to contact the vertebral surface of the passage. Additionally, the generally rectangular member could be curved and the device could expand by straightening the generally rectangular member such that the plurality of ribs and/or the generally rectangular member contact and frictionally engage the vertebral surface of the passage.

In yet another embodiment, the invention includes an alternative medical device for implantation into a spine of a patient having two generally rectangular members. The medical device includes first and second generally rectangular members each having first and second longitudinal edges and a plurality of ribs extending between and connecting the first and second longitudinal edges. The first and second generally rectangular members and plurality of ribs are composed of an elastic or shape-memory material and the device is expandable from a contracted state to an expanded state.

In the above-described device, the contracted and expanded states can be described in various ways. For instance, a distance between a mid-point of a rib connecting the first longitudinal edges of the first and second generally rectangular member and a mid-point of a corresponding rib connecting the second longitudinal edges is smaller in the contracted state than in the expanded state. Alternatively, the first and second generally rectangular members and the plurality of ribs define a volume and the defined volume is smaller in the contracted state than in the expanded state.

The plurality of ribs may further have a projection, such as a tine, tooth, or barb, on an outer surface of a rib to aid in frictionally engaging the vertebral surface. The plurality of ribs may be composed of nitinol or another shape memory material. Additionally, where each of the first and second generally rectangular members and the plurality of ribs have a thickness, the thickness of the first and second generally rectangular members are larger than the thickness of the plurality of ribs.

In use, the device described above can be used to repair a disc having a herniated nucleus pulposus, wherein the disc is situated between a cranial and caudal vertebra. A passage is formed in the cranial vertebra that extends into the disc using an angled anterior approach, such as an anterior lateral approach, that enters the cranial vertebra and terminates at a posterior region of the disc that includes the herniated nucleus pulposus. A device having first and second generally rectangular members each having first and second longitudinal edges and a plurality of ribs extending between and connecting the first and second longitudinal edges of the first and second generally rectangular members is placed into the passage. The device is expanded to frictionally engage the vertebral surface of the passage.

The device can expand using various techniques. Where the device is composed of a shape memory material, the device can automatically expand as a result of a temperature change after it is located in the passage. Where the device is composed of an elastic material, the device can expand as a result of expanding or inflating a balloon located between the plurality of ribs such that the ribs are pushed outward to contact the vertebral surface of the passage. Additionally, the first and second generally rectangular members could be curved and the device could expand by straightening the first and second generally rectangular members such that the plurality of ribs and/or the first and second generally rectangular member contact and frictionally engage the vertebral surface of the passage.

### BRIEF DESCRIPTION OF THE FIGURES

FIG. 1A is an axial cross section through a portion of the cervical spine.

FIG. 1B is an anterior view of a portion of the cervical spine.

FIG. 2A is an axial cross section of a portion of the cervical spine and an interbody bone graft.

FIG. 2B is an anterior view of a portion of the cervical spine with a bone graft.

FIG. 2C is an anterior view of a portion of the cervical spine with a plate attached with screws to the surrounding vertebrae.

FIG. 2D is a partial sagittal cross section of a portion of the cervical spine and the fusion system of FIG. 2C.

FIG. 3A is an axial cross section of the neck with a guide wire inserted into the disc.

FIG. 3B is a sagittal cross section of a portion of the neck depicted in FIG. 3A.

FIG. 3C is an axial cross section of the neck with a tissue dilator passed over the guide wire.

FIG. 3D is a sagittal cross section of a portion of the spine and the embodiment of the invention drawn in FIG. 3C.

FIG. 3E is an axial cross section of the neck with a sleeve passed over the tissue dilator.

FIG. 3F is an axial cross section of the neck with a guide wire disposed within the passageway created by the sleeve.

FIG. 3G illustrates an alternative embodiment of the invention with a grasping tool inserted into the disc space.

FIG. 3H illustrates an alternative embodiment of the invention wherein a flexible tip guide wire is inserted into the disc space.

FIG. 3I illustrates an alternative embodiment of the invention wherein a suture anchor has been inserted into a surrounding vertebra.

FIG. 3J illustrates an anterior view of the embodiment of FIG. 3I.

FIG. 3K illustrates a sagittal cross section of a disc reconstruction device being inserted between two vertebrae.

FIG. 3L is a sagittal cross section showing the disc reconstruction device placed into the disc.

FIG. 3M illustrates a suture from the anchor fastened across the anterior position of the discs reconstruction device.

FIG. 3N is an axial cross section of the neck with the disc reconstruction device in position.

FIG. 3O is an anterior view of a portion of the spine illustrating how two ends of a suture, from the suture anchor, have been fastened to each other.

FIG. 3P shows an alternative embodiment wherein the disc reconstruction device has been fastened to the disc.

FIG. 4A is a lateral view of a disc reconstruction device.

FIG. 4B is an axial cross section of the embodiment of the invention drawn in FIG. 4A.

FIG. 4C is a lateral view of an alternative embodiment of the disc reconstruction device which is generally cylindrical in shape.

FIG. 4D is an axial cross section of an alternative embodiment of a disc reconstruction device.

FIG. 4E is an axial cross section of an alternative embodiment in the form of a sheet of mesh which has been folded and fastened to increase the surface area of the device.

FIG. 4F is an axial cross section of an alternative embodiment including a lumen.

4G is an axial cross section of an alternative embodiment in the form of a hollow device that has a larger surface area.

FIG. 5A is an axial cross section of the spine and an alternative embodiment of the invention including an instrument used to remove bone spurs.

FIG. 5B is an oblique view of the end of a cutting tool.

FIG. 5C is an axial cross section of the spine with a reconstructed disc.

FIG. 6 is an oblique view of the tip of a tool that may be used to extract disc tissue.

FIG. 7A is a lateral view of an alternative embodiment of a tool that may be used to extract disc tissue.

FIG. 7B is a lateral view of the embodiment of the invention drawn in FIG. 7A, with the tips of the device depicted in a bent configuration.

FIG. 7C is a lateral view of an embodiment of the invention, wherein the tips of the pinching component have been forced together by advancing the sleeve.

FIG. 8A is a longitudinal cross section of the tip of an alternative embodiment of the invention that includes a cutting tool located within a sleeve.

FIG. 8B is an end view of the tip of the embodiment of the invention drawn in FIG. 8A.

FIG. 9A is a partial sagittal cross section of the spine and an alternative embodiment of the invention showing a guide wire placed through a portion of a vertebra and into a disc.

FIG. 9B is a partial sagittal cross section of the spine that has been reconstructed with a device that promotes bone in-growth into one portion of the device and soft tissue in-growth into another portion of the device.

FIG. 9C is a lateral view of the device that was inserted into the reconstructed spine drawn in FIG. 9B.

FIG. 9D is a sagittal cross section of an alternative embodiment of the invention wherein a mesh material surrounds a dowel-shaped piece of bone and disc component.

FIG. 10A is an anterior view of a portion of the spine with radiopaque markers embedded in a vertebra.

FIG. 10B is a lateral view of a portion of the spine with radiopaque markers embedded in a vertebra.

FIG. 10C is an anterior view of a portion of the spine with in instrument being inserted between the radiopaque markers.

FIG. 10D is a lateral view of a portion of the spine and the embodiment of the invention drawn in FIG. 10D.

FIG. 11A is a lateral view of partial sagittal cross section of a portion of the spine and an alternative implant.

FIG. 11B is an exploded lateral view of the implant pictured in FIG. 11A.

FIG. 11C is a longitudinal cross section of the implant drawn in FIG. 11A.

FIG. 11D is an end view of the implant pictured in FIG. 11A.

FIG. 12A is a lateral view of a partial sagittal cross section of the spine having a hole drilled though the vertebra.

FIG. 12B is a lateral view of a partial sagittal cross section of the spine with the soft tissue and attached elongate member disposed in the hole in the vertebra.

FIG. 12C is a lateral view of a partial sagittal cross section of the spine with the soft tissue and attached elongate member disposed in the hole in the vertebra, with the elongate threaded member disposed about the elongate member.

FIG. 12D is a lateral view of a partial sagittal cross section of the spine and the embodiment of the invention drawn in FIG. 11C.

FIG. 13A is a lateral view of a partial sagittal cross section of the spine having a tunnel extending through the vertebra and ending in the spinal canal.

FIG. 13B is a lateral view of a partial sagittal cross section of the spine having a tunnel extending through the vertebra and a portion of the anulus fibrosus and ending in the spinal canal.

FIG. 14A is a lateral view of a partial sagittal cross section of the spine and an alternative embodiment of the invention having a distal end that radially expands.

FIG. 14B is an exploded lateral view of the embodiment of the invention drawn in FIG. 14A.

FIG. 14C is a view of distal end 442 of the elastic/shape memory component drawn in FIG. 14B.

FIG. 14D is a view of the distal end 442 of the elastic/shape memory component drawn in FIG. 14C.

FIG. 14E is a lateral view of the elastic/shape memory component drawn in FIG. 14B and a sleeve.

FIG. 14F is a lateral view of the embodiment of the invention drawn in FIG. 14E.

FIG. 14G is a lateral view of a partial sagittal cross section the spine and the embodiment of the invention drawn in FIG. 14A inserted into a tunnel in a vertebra.

FIG. 14H is a lateral view of a partial sagittal cross section of the spine and the embodiment of the invention drawn in FIG. 14G with the sleeve retracted.

FIG. 14I is a lateral view of a partial sagittal cross section of the spine and the embodiment of the invention drawn in FIG. 14H with a clamp about the elongate member to secure the elongate threaded members.

FIG. 14J is a lateral view of a partial sagittal cross section of the spine and an alternative embodiment of the invention drawn in FIG. 14A.

FIG. 15A is a lateral view of a partial sagittal cross section of the spine having an alternative implant having a covering over the expandable distal end.

FIG. 15B is an exploded lateral view of the embodiment of the invention drawn in FIG. 15A.

FIG. 15C is a lateral view of a longitudinal cross section of the embodiment of the invention drawn in FIG. 15A.

FIG. 16A is a lateral view of a partial sagittal cross section of the spine having an alternative implant, wherein the distal end has an expandable spiral or coil.

FIG. 16B is a view of the distal end of the embodiment of the invention drawn in FIG. 16A.

FIG. 16C is a view of the end of the distal end of the embodiment of the invention drawn in FIG. 16B. The end of the device has expanded radially.

FIG. 17A is a lateral view of an alternative embodiment of the invention having an alternative implant positioned in the vertebra and disc, wherein the distal end can expand radially.

FIG. 17B is a lateral view of the embodiment of the invention drawn in FIG. 17A with expandable distal end 462 of the soft tissue in-growth component drawn in its expanded configuration.

FIG. 17C is a lateral view of an alternative embodiment of the invention drawn in FIG. 17A.

FIG. 17D is a lateral view of the embodiment of the invention drawn in FIG. 17C, with distal end 462 drawn in its expanded configuration.

FIG. 18A is a lateral view of a portion of the spine wherein the disc is herniated or protrudes posterior to the vertebrae into the spinal canal.

FIG. 18B is a lateral view of a partial sagittal cross section of a portion of the spine and a drill bit.

FIG. 18C is a lateral view of a partial sagittal cross section of a portion of the spine and an alternative embodiment of the invention having a cannulated drill bit advanced over a guidewire.

FIG. 18D is a lateral view of a partial sagittal cross section of a portion of the spine and an alternative embodiment of the invention wherein a mill tip is used to create a tunnel.

FIG. 18E is an axial cross section through a disc with a drill bit sitting within the anulus fibrosus and protruding into the spinal canal.

FIG. 18F is an axial cross section through a disc and a mill tip sitting within a tunnel.

FIG. 19A is a lateral view of a partial sagittal cross section of the spine and an alternative embodiment.

FIG. 19B is an oblique view of the proximal end of the device drawn in FIG. 19A.

FIG. 19C is an axial cross section of cranial vertebra 120 with device 472 located within the vertebra.

FIG. 19D is an axial cross section of a disc and the device, which lies along the edge of the surgically treated disc.

FIG. 19E is an oblique view of the distal end of the device of FIG. 19A.

FIG. 20A is a lateral view of an alternative embodiment of the invention that can be inserted into a vertebra and a disc.

FIG. 20B is a view of the proximal end of the embodiment of the invention drawn in FIG. 20A.

FIG. 20C is a view of the inferior surface of the embodiment of the invention drawn in FIG. 20A.

FIG. 20D is a view of the proximal end of the embodiment of the invention drawn in FIG. 20B and a balloon in a deflated configuration.

FIG. 20E is a view of the proximal end of the embodiment of the invention drawn in FIG. 20D and a balloon in an expanded configuration.

FIG. 20F is a lateral view of a sagittal cross section through a portion of the spine and a lateral view of the embodiment of the invention drawn in FIG. 20A.

FIG. 20G is a lateral view of a partial sagittal cross section of a portion of the spine and a lateral view of the embodiment of the invention drawn in FIG. 20F with the spine drawn in a flexed position.

FIG. 20H is an axial cross section through a disc and the embodiment of the invention drawn in FIG. 20G.

FIG. 20I is an axial cross section of cranial vertebra 120 and the device drawn in FIG. 20G.

FIG. 20J is an axial cross section of a vertebra and an alternative embodiment of the device.

FIG. 20K is an axial cross section of a vertebra having a tunnel with a different cross section and the device of FIG. 20K.

FIG. 20L is an axial cross section of a vertebra and an alternative embodiment of the invention having a curved spine.

FIG. 20M is an axial cross section of a vertebra having a tunnel with a rectangular cross-section and an alternative embodiment of the invention.

FIG. 20N is an axial cross section of a vertebra having a tunnel with a circular cross-section and the device depicted in FIG. 20M in its contracted configuration.

FIG. 20O is an axial cross section of a vertebra having a tunnel with a circular cross-section and the device depicted in FIG. 20M in its expanded configuration.

FIG. 21 is a view of the proximal end of an alternative embodiment of the invention drawn in FIG. 20A.

### DETAILED DESCRIPTION OF THE INVENTION

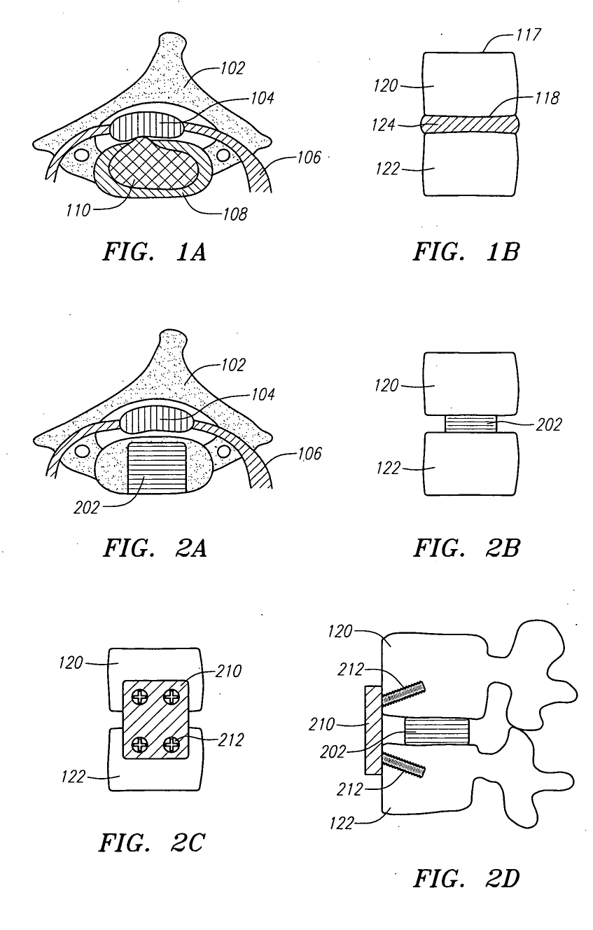

FIG. 1A is an axial cross section through a portion of the cervical spine, which shows cervical vertebra 102, spinal cord 104, nerves 106, anulus fibrosus (AF) 108, and nucleus pulpous (NP) 110. The disc is herniated. In other words, nucleus pulposus 110 extends through a defect in anulus fibrosus 108. The herniated nucleus pulpous (HNP) is shown pressing against spinal cord 104. FIG. 1B is an anterior view of a portion of the cervical spine, depicting disc 124 surrounded by cranial and caudal vertebrae 120, 122. Each vertebra has an upper vertebral endplate 117 and a lower vertebral endplate 118.

FIG. 2A is an axial cross section of a portion of the cervical spine and interbody bone graft 202. The drawing illustrates an anterior cervical discectomy and fusion. Bone graft 202 is placed into the disc space after the disc has been removed. FIG. 2B is an anterior view of a portion of the cervical spine, which illustrates cranial and caudal vertebrae 120, 122 surrounding bone graft 202. FIG. 2C is an anterior view of a portion of the cervical spine. A plate 210 and four screws 212 have been fastened to the surrounding vertebrae 120, 122. This fixation facilitates spinal fusion by eliminating movement between the surrounding vertebrae 120, 122. FIG. 2D is a partial sagittal cross section of a portion of the cervical spine and the fusion system drawn in FIG. 2C.

### Removal of Herniated Nucleus Pulposus and/or Bone Spurs

Access through the Disc

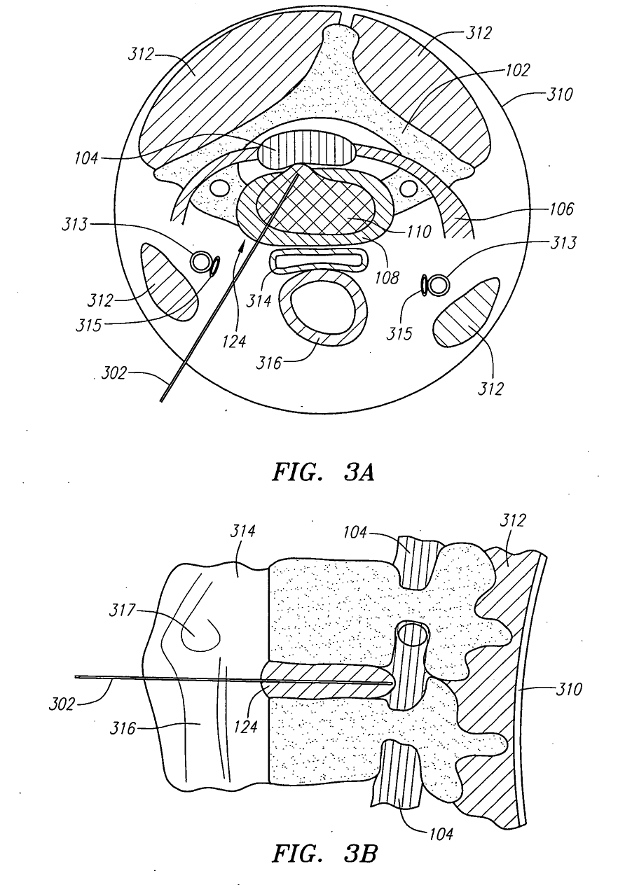

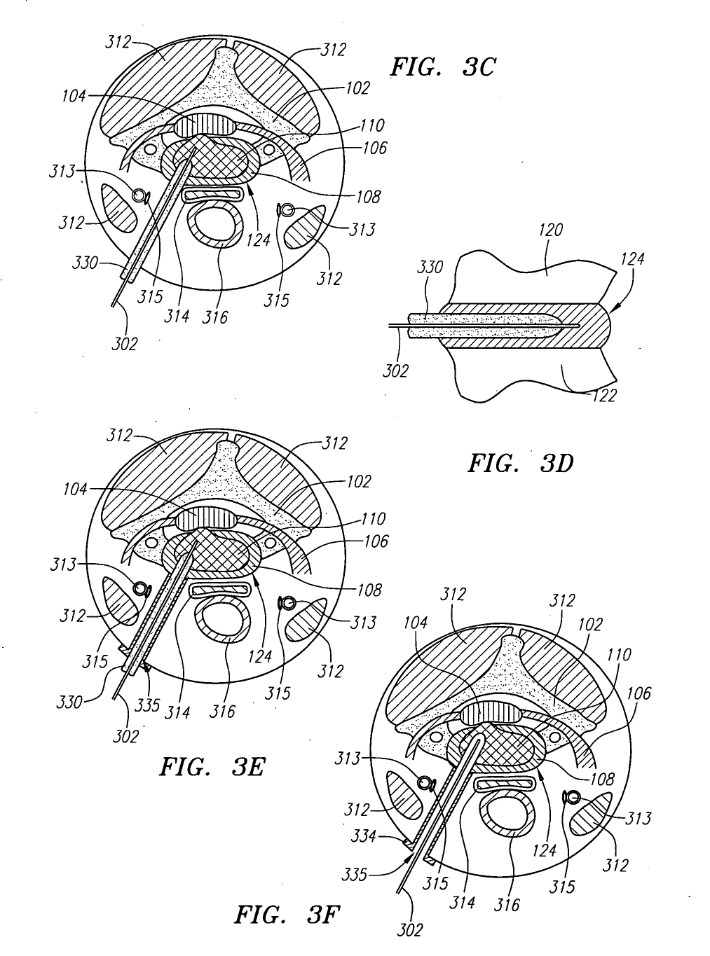

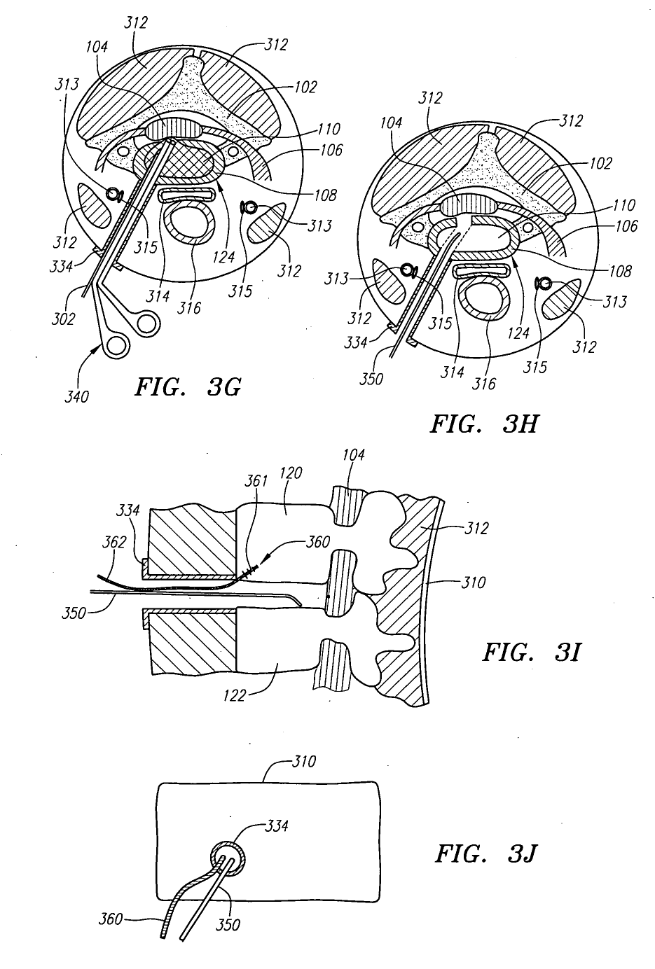

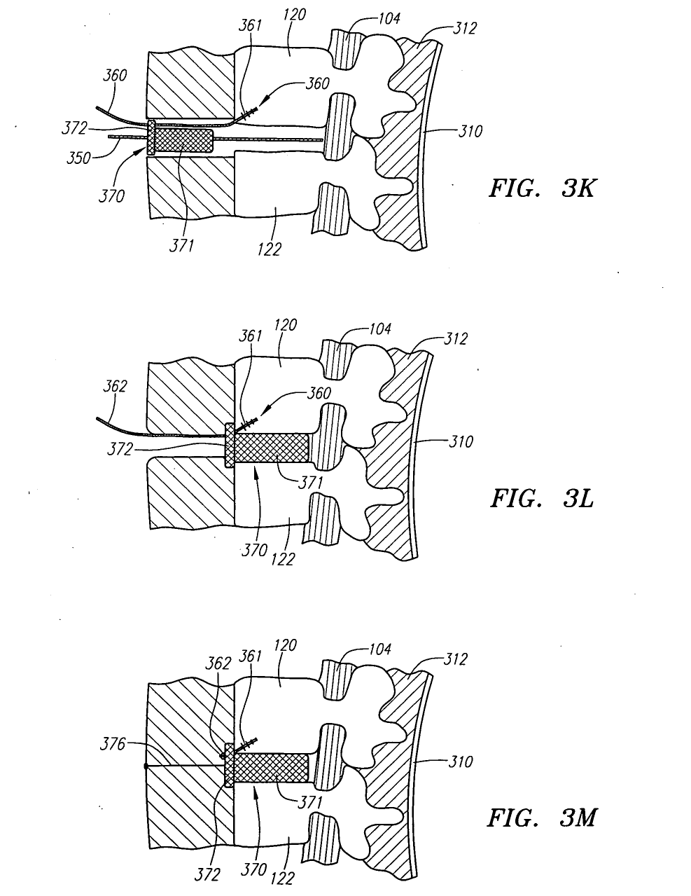

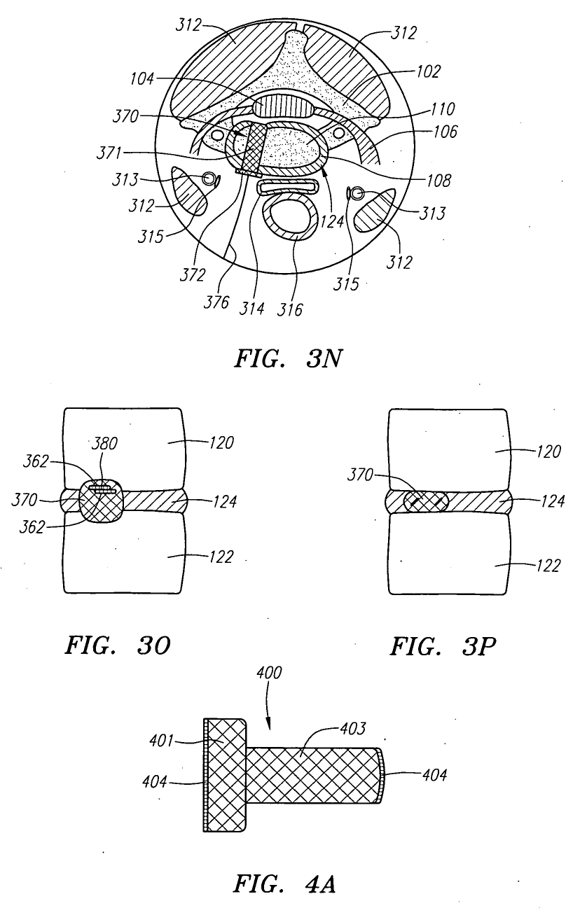

FIG. 3A is an axial cross section of the neck including skin 310 and muscles 312. Esophagus 314 lies directly anterior to the disc, which is made up of anulus fibrosus 108 and nucleus pulposus 110. Trachea 316 lies just anterior to esophagus 314. The herniated nucleus pulpous (HNP) is shown pressing against spinal cord 104. The herniated nucleus pulposus may lie anterior to the posterior longitudinal ligament (PLL) (not shown). Alternatively, the herniated nucleus pulposus may extend through a defect in the posterior longitudinal ligament. FIG. 3B is a sagittal cross section of a portion of the neck and the embodiment of the invention drawn in FIG. 3A. Spinal cord 104 is shown posterior to disc 124. Esophagus 314 is anterior to the vertebrae and lies between larynx 317 and trachea 316 and the spine.

Guide wire 302 has been inserted into the region of the herniated nucleus pulpous. Guide wire 302 may be passed through a blunt cannulated instrument (not shown). The blunt instrument may be used to create safe path to the spine using an anterior lateral approach (from the left or right side). The side to side obliquity prevents a patient's head or chest from obstructing the path of instruments inserted into the spine. Additionally, it minimizes the risk of injury to the midline structures such as the trachea, larynx, and esophagus. Guide wire 302 may pass through the blunt instrument and into the disc. Image guidance enables the physician to direct guide wire 308 into the herniated nucleus pulpous. Image guidance also helps the physician avoid injuring the surrounding structures such as esophagus 314, carotid artery 313, and internal jugular vein 315. MRI, CT, Fluoroscopy, and/or navigation systems may be used as available or desired. For instance, the images obtained during CT guidance may be used as reference points for the navigation system. The navigation system enables the physician to use real-time, virtual CT during a portion of the procedure. Additionally, the patient could be given oral contrast to help the physician identify the esophagus. Alternatively, a device could be inserted into the esophagus to help the physician identify the esophagus during imaging. An instrument that uses information from the CT scan to guide the guide wire to a specific location using robotics may also be used. Standard software may be used to reconfigure the axial CT images into sagittal cross sections and images of the exterior of the spine. The reconfigured images create CAD-like models. Image analysis software may also be used to convert the CT data into a CAD (Computer assisted drawing/drafting) format. The interventionalist may view the spinal images on a LED monitor. The interventionalist may use CAD software to determine the optimal angle of guide wire insertion, depth of guide wire insertion, and diameter of opening created into the disc. The CAD information may be used to guide a CAM (computer assisted manufacturing) tool. Use of CAD/CAM technology minimizes injury to the disc and surrounding structures. Contrast could be injected into the disc (discogram) and/or into the subdural space (myleogram) to help visualize the herniated nucleus pulposus. CAD/CAM technology could be used to guide and/or insert instruments and devices into and out of the spine.

FIG. 3C is an axial cross section of the neck and the embodiment of the invention drawn in FIG. 3A. Tissue dilator 330 has been passed over the guide wire 302. Tissue dilator 330 enlarges the passage to and into disc 124. Tissue dilator 330 displaces, rather than removes, disc tissue. The tip of tissue dilator 330 could have retractable cutting surfaces (not shown). The cutting surfaces, or blades, could be used to cut a hole in anulus fibrosus 108. FIG. 3D is a sagittal cross section of a portion of the spine and the embodiment of the invention drawn in FIG. 3C.

FIG. 3E is an axial cross section of the neck and the embodiment of the invention drawn in FIG. 3C. Sleeve 334 has been placed over tissue dilator 330, thereby providing port 335 for passing instruments into and out of the incision in the skin 310. A speculum-like retractor (not shown) may be used rather than the sleeve in alternative embodiments of the invention. FIG. 3F is an axial cross section of the neck and the embodiment of the invention drawn in FIG. 3E. The tissue dilator has been removed and guide wire 302 rests within a hole created in disc 124.

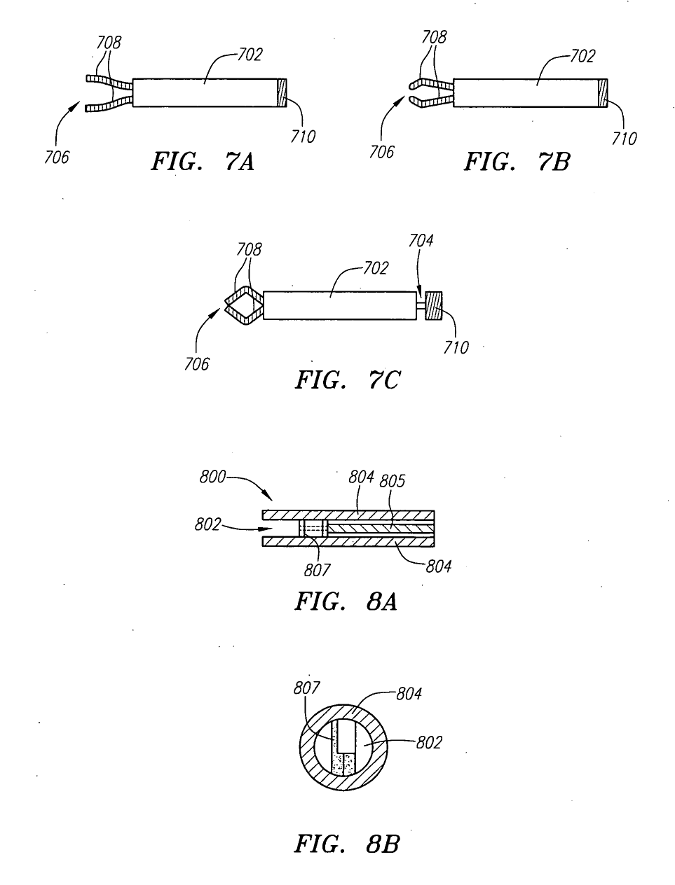

In FIG. 3G, “grasper” instrument 340 has been placed next to guide wire 302. Grasper instrument 340 may be used to remove the herniated nucleus pulposus110 using image guidance as desired or as necessary. Additional methods and devices could be used to remove herniated nucleus pulposus110. For example, Chymodiactin, chondroitinase ABC, or other material including electrocautery or radiofrequency devices could be injected into herniated nucleus pulposus110 to dissolve herniated nucleus pulposus110 or a laser or other device may be used to desiccate or shrink herniated nucleus pulposus110. Mechanical devices such as brushes may used to remove herniated nucleus pulposus110. Alternatively, devices such as those described in FIGS. 6, 7A-C, and 8A-B may be used to remove the herniated nucleus pulposus. Precise location of instruments within the herniated nucleus pulposus enables the inventionalist to deliver small doses of enzymes, such as Chymodiactin or chondroitinase ABC, directly into the herniated nucleus pulposus. Previously, large doses of these enzymes have been injected into the center of the disc rather than into the herniated nucleus pulposus. This has resulted digest the entire NP as well as the HNP. Precise location of the instruments within the HNP also enables the inventionalist to remove only the HNP with the other methods listed (such as radiofrequency).

FIG. 3H is an axial cross section showing how the original guide wire has replaced with flexible tip guide wire 350. Flexible tip guide wire 350 helps prevent injuring spinal cord 104.

In FIG. 3I, a fastener, such as suture anchor 360 comprising a screw 361 and coupled suture 362, has been inserted into the cranial vertebra 120. Suture 362 is coupled to screw 361 such that two free ends of the suture can pass through the skin incision. Alternatively, the suture may be coupled to the screw such that only one free end passes through the skin incision. Two or more suture anchors may be inserted into each vertebrae. Screw portion 361 of suture anchor 360 is preferably resorbable. FIG. 3J is an anterior view that illustrates guide wire 350 and suture 360 exiting port 335 created by sleeve 334.

In FIG. 3K, suture 362 from suture anchor 360 has been passed through disc reconstruction device 370. Disc reconstruction device 370 has also been placed over guide wire 350 and suture 362. A detailed description of disc reconstruction device 370 is provided in a later section.

FIG. 3L is a sagittal cross section showing disc reconstruction device 370 placed into the disc. One of the free ends of suture 362 is seen extending out of the body. In FIG. 3M, suture 362 from anchor 361 has been fastened across the enlarged anterior portion 372 of the disc reconstruction device 370. The free ends of suture 362 could be welded to disc reconstruction device 370. Alternatively, where two or more suture anchors have been inserted into the vertebrae, the ends of the sutures could be ultrasonically welded to each other. Alternatively, fastening components could be crimped over the ends of the suture or sutures. The ends of the sutures could also be tied over the disc reconstruction device. Other fastening mechanisms may be used to fasten the disc reconstruction device to the disc and/or the vertebra. The thin line 376 anterior to the disc reconstruction device represents the path, through the soft tissues, used in accordance with the invention. FIG. 3N is an axial cross section of the neck with disc reconstruction device 370 inserted within disc 124.

FIG. 3O is an anterior view of a portion of the spine illustrating how the two ends of suture 362, from the anchor, have been fastened to each other at 380. FIG. 3P shows an alternative embodiment wherein disc reconstruction device 370 has been fastened to disc 124. Staples, sutures, or other anchors could be used to fasten disc reconstruction device 370 to disc 124. Disc reconstruction device370 may be fastened to the vertebra or vertebrae and/or the disc in alternative embodiments of the invention.

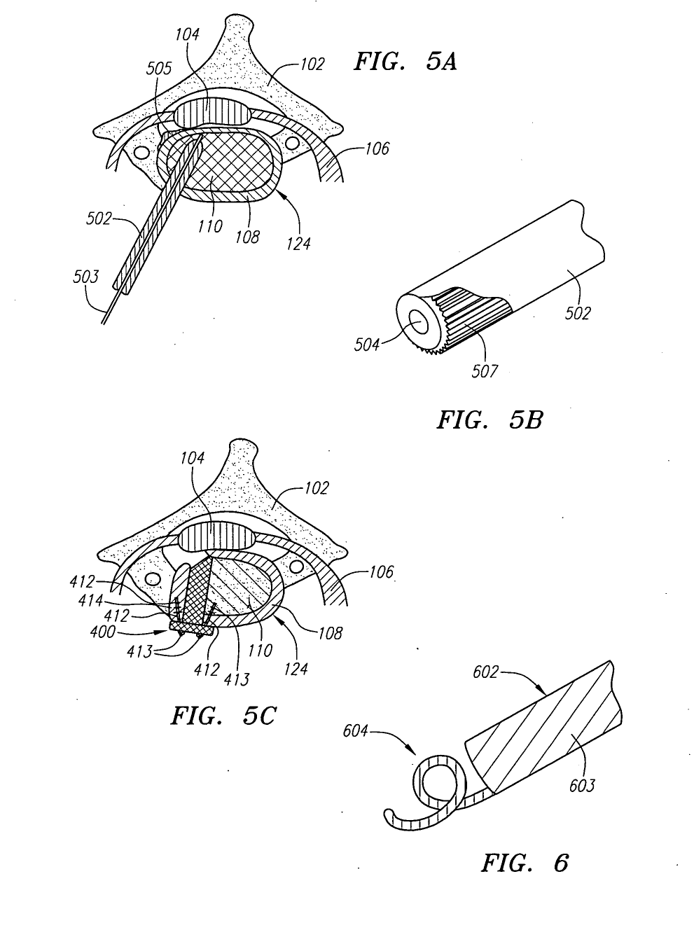

FIG. 5A is an axial cross section of the spine and an instrument that used to remove bone spurs 505, thus enlarging the neuroforamen (the area that surrounds the spinal nerve, which is formed by the vertebrae directly cranial and caudal to the disc and the posterior-lateral portion of the disc). The instrument includes cutting tool 502 placed over guide wire 503. Cutting tool 502 may include a bur, reamer, or drill bit. Cutting tool 502 preferably oscillates or vibrates rather rotating 360 degrees. Cutting tool 502 may be connected to power tools. FIG. 5B is an oblique view of the end of cutting tool 502 described in the text with reference to FIG. 5A. Cutting tool 502 is cannulated, i.e., includes lumen 504, to fit over guide wires. Cutting tool 504 includes serrated portion 507 at the distal end to aid in cutting and/or drilling. Cutting tool 504 can be guided by CAD/CAM technology. The position of the cutting tool can be checked, even repeatedly, by radiographic studies such as CT scans. The CAM system preferably has technology that enables the system to quantify the amount of pressure on the tip of the cutting tool. The CAM system immediately stops movement (rotation and advancement) of the cutting tool when the pressure on the tip of the cutting tool falls. For example, the movement of the cutting tool could stop if the pressure falls below a preset amount (e.g. 1 psi, alternatively about 2 psi, alternatively about 3 psi, alternatively about 4 psi, alternatively about 5 psi, alternatively about 6 psi, alternatively about 7 psi, alternatively about 8 psi, alternatively about 10 psi, alternatively about 12 psi, alternatively about 14 psi, alternatively about 16 psi, alternatively about 18 psi, alternatively about 20 psi, alternatively about 22 psi, alternatively about 24 psi, alternatively about 26 psi, alternatively about 28 psi or more) or falls a certain percent (e.g. about 10%, alternatively about 12%, alternatively about 14%, alternatively about 16%, alternatively about 18%, alternatively about 20%, alternatively about 22%, alternatively about 24%, alternatively about 26%, alternatively about 28% or more). The technology prevents undesirable advancement of the cutting tool as the tip of the tool cuts through the final millimeter of bone or soft tissue.

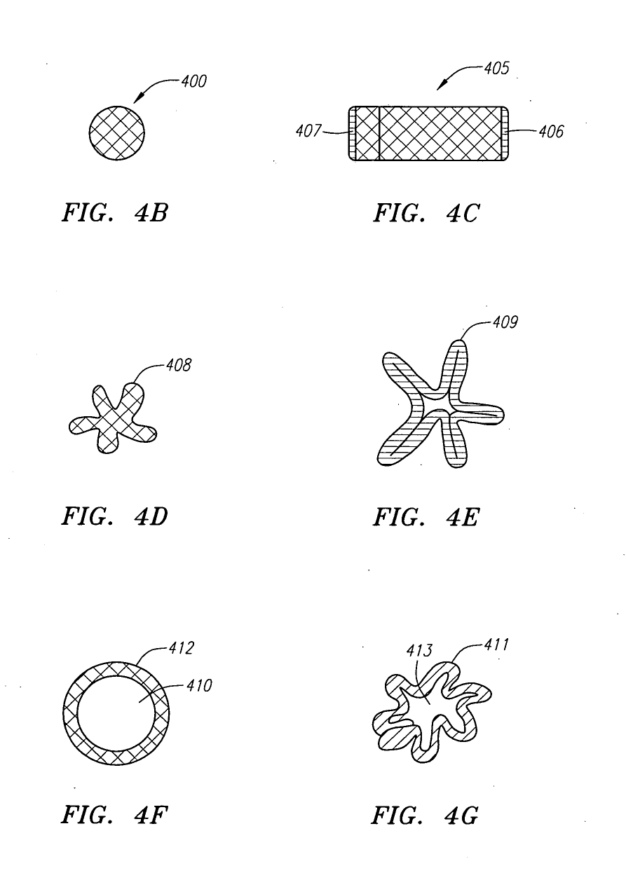

FIG. 5C is an axial cross section of the spine showing how the disc has been reconstructed with the embodiment of the invention drawn in FIG. 4A. The cross-section includes cervical vertebra 102, spinal cord 104, nerves 106, and disc 124 including anulus fibrosus (AF) 108 and nucleus pulpous (NP) 110. The bone spurs have been removed using the cutting tool as previously described. Disc reconstruction device 400 has been inserted into disc 124 using an anterior lateral approach (from the left or right side). A right sided approach is preferably used to treat left sided disc pathology and a left sided approach is preferably used to treat right sided disc pathology. As stated previously, the side to side obliquity prevents a patient's head or chest from obstructing the path of instruments inserted into the spine. Additionally, it minimizes the risk of injury to the midline structures such as the trachea, larynx, and esophagus. Disc reconstruction device 400 is then attached to disc 124 using anchors 412. Anchors 412 have elongate member 414 with enlarged ends 413. In one embodiment, enlarged ends 413 may have a transverse component that is substantially perpendicular to a longitudinal axis of elongate member 414.

Access through the Vertebra

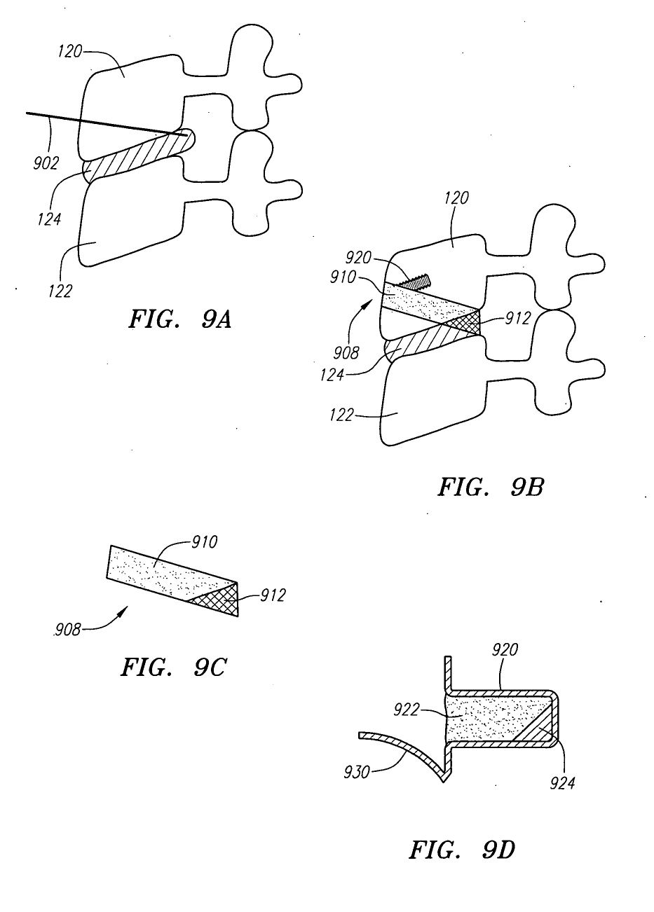

Injuries in the disc can also be accessed through the surrounding vertebrae. Because bone heals better than the disc tissue, accessing the pathology through the surrounding vertebrae minimize the injury to the disc. In order to minimize injury to other intervertebral discs, instruments are preferably inserted through the cranial or caudal vertebrae that lie adjacent to the injured disc and do not pass through any other intervertebral discs. FIG. 9A is a partial sagittal cross section of the spine and an alternative embodiment of the invention. Guide wire 902 has been placed through a portion of cranial vertebra 120 and into a posterior portion of disc 124. Guide wire 902 is preferably placed through cranial vertebra 120 under image guidance using an anterior lateral approach (from the left or right side). As stated previously, the side to side obliquity prevents a patient's head or chest from obstructing the path of instruments inserted into the spine. Additionally, it minimizes the risk of injury to the midline structures such as the trachea, larynx, and esophagus. Cannulated drill bits or reamers may then be used to create a hole through vertebra 120. Tools as described in other embodiments of the invention may be used to remove the herniated nucleus pulposus or bone spurs. This embodiment of the invention helps prevent disc damage like certain embodiments disclosed in U.S. Pat. No. 6,878,167, which is hereby incorporated by reference in its entirety.

FIG. 9B is a partial sagittal cross section of the spine showing how the herniated nucleus pulposus has been removed. The spine has been reconstructed with device 908, which includes bone in-growth section 910 and soft tissue in-growth section 912. Screw 920 has been passed through the device and the vertebra to secure the device in place.

FIG. 13A is a lateral view of a partial sagittal cross section of the spine. Tunnel 126 terminates in spinal canal 129 cranial to intervertebral disc 124 and vertebral endplate of the cranial vertebra 120. Alternatively, tunnel 126 could be placed in caudal vertebra 122. Tunnel 126 preferably ends within the herniated nucleus pulposus and just anterior to the posterior longitudinal ligament (not drawn). The posterior longitudinal ligament can be cut with the tip of sharp instrument. The tip of the sharp instrument is preferably about 1-2 mm long. The instruments used to create the tunnel, incise the posterior longitudinal ligament, remove the herniated nucleus pulposus, and insert the device are preferably guided by the previously described CT image/CAD/CAM technology.

FIG. 13B is a lateral view of a partial sagittal cross section of the spine and an alternative embodiment of the invention. Tunnel 126 terminates in spinal canal 129 and in anulus fibrosus 108 of intervertebral disc 124. Tunnel 126 preferably does not enter the nucleus pulposus 110 portion of intervertebral disc 124. Alternatively, the tunnel may enter the outer-most 1 to 5 mm of the intervertebral disc. In another embodiment, the tunnel could end in the nucleus pulposus of the intervertebral disc or end in the anulus fibrosus and the nucleus pulposus of the intervertebral disc.

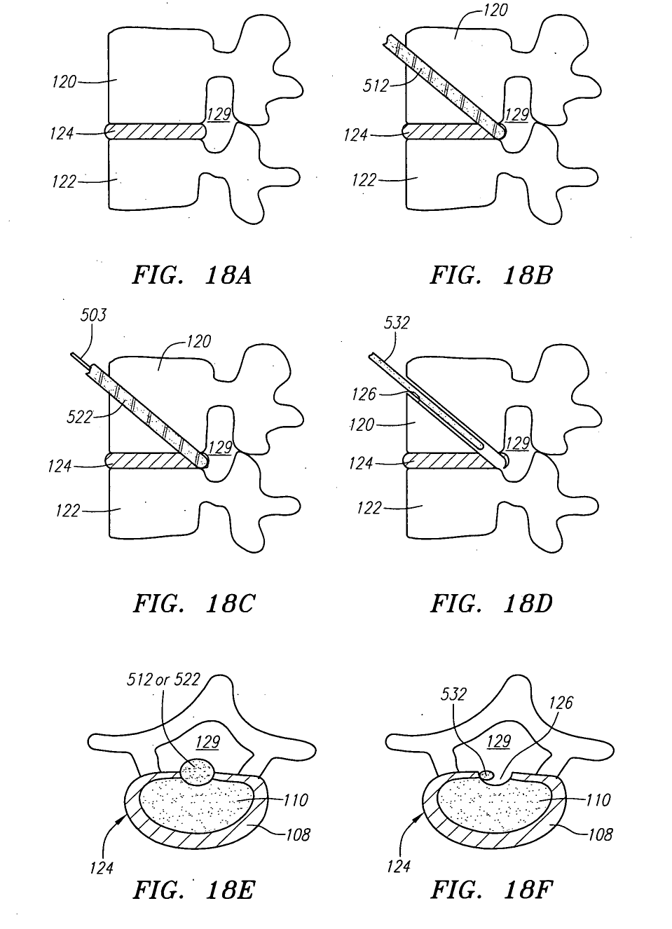

FIG. 18A is a lateral view of a portion of the spine. Disc 124 is herniated or protrudes posterior to vertebrae 120, 124 into spinal canal 129. FIG. 18B is a lateral view of a partial sagittal cross section of a portion of the spine and drill bit 512. Drill bit 512 has cutting flutes around a pointed distal tip. Drill bit 512 extends through vertebra 120, into disc 124 and into the herniated nucleus pulposus. Drill bit 512 is preferably guided by the previously described CT image/CAD/CAM technology or a robot that has features that prevent advancement of drill bit 512 when the drill encounters less pressure. The feature prevents excessive advancement of drill bit 512 into spinal canal 129. Excessive advancement of drill bit 512 could injure the spinal cord. The tip of drill bit 512 experiences less pressure as it advances through disc material than when it advances through the vertebra. The robot preferably has features than control the depth of drill bit advancement. For example, the robot could be programmed to advance the drill bit about 10 mm, alternatively about 12 mm, alternatively about 14 mm. The distance between the tip of the partially advanced drill bit and the herniated nucleus pulposus or spinal canal 129 could be measured from imaging studies. The robot could be programmed to advance the drill bit the predetermined distance. The measurements and drill bit advancements could be made several times. Drill bit 512 is advanced through a sleeve (not shown) in the soft tissues of the neck, similar to sleeve 334 drawn in FIG. 3E.

The tunnel preferably enters the cranial vertebra about 1 to 3 mm below the cranial vertebral endplate of cranial vertebra 120. Alternatively, the tunnel could enter the cranial vertebral endplate of cranial vertebra 120 or enter the cranial vertebra about 4 mm, alternatively about 5 mm, alternatively about 6 mm, or alternatively about 7 mm or more below the cranial vertebral endplate of cranial vertebra 120. The entry location is typically to the left or right of the mid-line of the vertebra. The entry location of drill bit 512 increases the angle between the drill bit and the longitudinal axis of the spine. The obliquity of the drill bit minimizes the cross sectional area of the tunnel at the vertebral endplate. The tunnel is preferably made through cranial vertebra 120. The intervertebral discs of the cervical spine are angled relative to the longitudinal axis of the body. The posterior portion of the disc is generally cranial to the anterior portion of the disc. The angle of the disc and the ability to move a patient's head from the area of drill insertion facilitate the formation of a tunnel in the cranial vertebrae.

Alternatively, the tunnel may be made through the vertebra caudal 122 to disc 124 or through the vertebrae cranial and caudal to the disc. Tunnels through caudal vertebra 122 are easier in the vertebrae cranial to the C4-C5 disc. Tunnels through the caudal vertebra 122 also facilitate excision of disc fragments that migrate caudal to the intervertebral disc 124. Tunnels through the caudal vertebra preferably start about 1 to 3 mm cranial to the caudal vertebral endplate of caudal vertebra 122. The distal end of the tunnels through the cranial vertebra preferably include about 1 to 3 mm of the posterior wall of the cranial vertebra. Alternatively, the tunnels may include about 4 mm, alternatively about 5 mm, or alternatively about 6 mm or more of the posterior wall of the vertebra. The tunnel may also extend completely anterior to the posterior wall of the vertebra. The distal end of tunnels through the caudal vertebra preferably include about 1 to 3 mm of the posterior wall of the caudal vertebra.

FIG. 18C is a lateral view of a partial sagittal cross section of a portion of the spine and an alternative embodiment of the invention. Cannulated drill bit 522 has been advanced over guidewire 503. The tip of drill bit 522 has cutting flutes. In contrast to the pointed drill bit tip depicted in FIG. 18B, the tip of drill bit 522 has a relatively flat tip that decreases protrusion of drill bit 522 into the spinal canal 129.

FIG. 18D is a lateral view of a partial sagittal cross section of a portion of the spine and an alternative embodiment of the invention. Tunnel 126, which extends through vertebra 120 and into disc 124, is milled, preferably under robotic control. Milling the tunnel enables surgeons, other physicians or technicians to create tunnels with minimal advancement of tools into the spinal canal. The tunnel is preferably, though not necessarily, circular in cross section. The diameter of the mill tip 532 is smaller than the diameter of tunnel 126. Mill tip 532 moves from side to side as it is advanced through vertebra 120.

FIGS. 18E and F are axial cross sections through disc 124. FIG. 18E depicts a drill bit, as depicted in FIGS. 18C or 18D, sitting within anulus fibrosus 108 and protruding into spinal canal 129. FIG. 18F is an axial cross section through disc124 and mill tip 532 drawn in FIG. 18D. Mill tip creates tunnel 126 that has a larger diameter than that of the mill tip without mill tip 532 protruding into spinal canal 129.

Radiopaque Markers

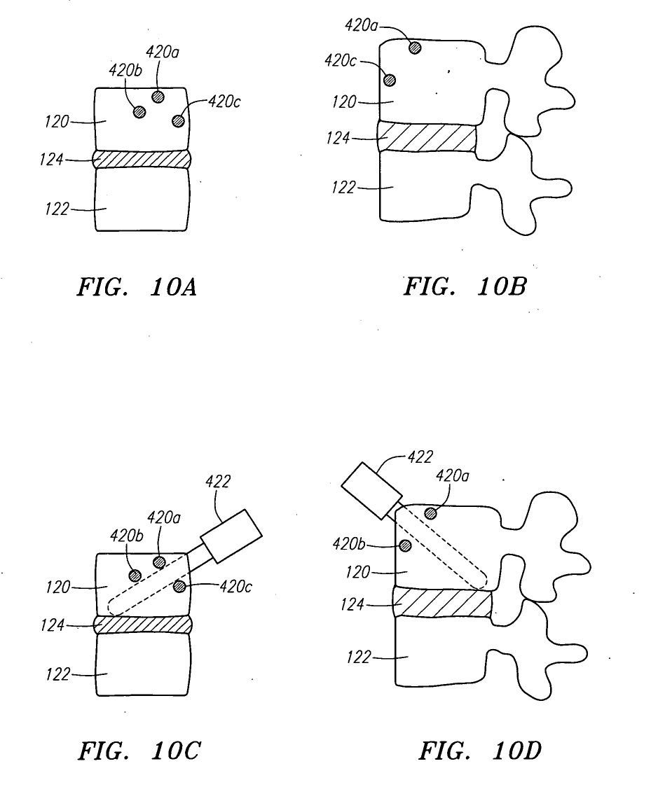

In any of the procedures described, where access is gained through either the disc or the surrounding vertebrae, radiopaque markers may be implanted into the surrounding vertebrae to aid the physician in positioning the guide wire, suture anchors, and/or implants. For example, as seen in FIGS. 10A-D, radiopaque markers can be used to guide instruments into the correct position.

A plurality of radiopaque markers can be placed into the surrounding vertebrae. FIG. 10A is an anterior view of a portion of the spine. FIG. 10B is a lateral view of a portion of the spine and the embodiment of the invention drawn in FIG. 10A. Three radiopaque markers 420a-c were placed into cranial vertebra 120. Radiopaque markers 420a-c could be threaded or impacted into the vertebra. Markers 420a-c are preferably about 1 to about 3 mm in diameter. The position of the markers is non-collinear, i.e., all of the markers do not lie or pass through the same straight line. The markers may be used to aid the physician or, alternatively, to guide a robot. The location of the markers relative to the spinal pathology, for example herniated nucleus pulposus, bone spurs, or a mass, may be determined during the surgical procedure by a CT scan, MRI scan, fluoroscopy, or other imaging modality. A computer may be used to control actuators or other tools that guide a wire, bur, drill, or other instrument to the spinal pathology. The software may repeatedly compare the location of the surgical instrument relative to the markers and the spinal pathology. Image guided navigation and a robot are used to minimize injury to the normal spinal tissues. Navigation and robotics enable the use of instruments with small diameters. The instruments are preferably about 1 to about 5 mm in diameter. Alternatively, the instruments could be about 6 mm, about 7 mm, about 8 mm, about 9 mm, or about 10 mm or larger in diameter. Navigation and robotics also enable precise placement of the instruments. The CT image/CAD/CAM technology guide the position and angle of tools to start the tunnel and the trajectory and movement of the instruments as the instruments are advanced through the vertebra and disc.

FIG. 10C is an anterior view of a portion of the spine and the embodiment of the invention drawn in FIG. 10A. Instrument 422 has been guided into the area of spinal pathology located near disc 124. The dotted lines indicate the portion of the tool within the vertebra. FIG. 10D is a lateral view of a portion of the spine and the embodiment of the invention drawn in FIG. 10D.

### Tools to Extract Tissue and Bone

FIG. 6 is an oblique view of the tip of a tool 602 that may be used to extract disc tissue. Tool 602 includes an elongate member and one or more corkscrews 604 located at the distal end of elongate member 603. Tool 602 may be advanced into the herniated nucleus pulposus under image guidance. Corkscrew 604 engages the herniated nucleus pulposus and tool 602 pulls the herniated nucleus pulposus away from the nerves as tool 602 is withdrawn from the patient.

FIG. 7A is a lateral view of an alternative embodiment of the invention related to that drawn in FIG. 6. Elongate instrument 706 includes sleeve 702 having a lumen and elastic pinching component 704 disposed within the lumen of sleeve 702. Elastic pinching component 704 comprises an elongate member having proximal and distal ends. The proximal end of elastic pinching component 704 has an enlarged area 710 that is at least as large as the diameter of sleeve 702 such that enlarged area 710 does not fit into the lumen of sleeve 702 and remains proximal of the proximal end of sleeve 702. The distal end of elastic pinching component 704 has at least two arms 708 that are adapted to be “pinched” together to grasp a material. Tips 708 of the pinching component can be advanced into the herniated nucleus pulposus under image guidance.

FIG. 7B is a lateral view of the embodiment of the invention drawn in FIG. 7A. Tips 708 of pinching component 704 have a bent configuration. Shape-memory materials, such as nitinol, may be used to manufacture the pinching component. Temperature change could cause the tips of the pinching component to transform into the bent position, as depicted. FIG. 7C is a lateral view of an embodiment of the invention wherein the tips 708 of the pinching component have been forced together by advancing the sleeve 702, or alternatively, by pulling elastic pinching component 704 proximally into sleeve 702. The tool may be used to grasp and remove disc tissue.

FIG. 8A is a longitudinal cross section of the tip of an alternative embodiment of the invention including device 800 having cutting tool 802 located within sleeve 804. Cutting tool 802 includes shaft 805 and cutting component 807 located at the distal end of shaft 805. The distal end of cutting tool 802 is preferably recessed within the lumen of sleeve 804. Shaft 805 of cutting tool 802 may have a smaller diameter than the lumen of sleeve 804. Device 800 may be connected to suction and/or a power tool such that tissue can be pulled into lumen of sleeve 804. Cutting component 807 could thereafter grind or morscelize tissue that is pulled into the tool. Small pieces of disc tissue may pass through the space between the shaft 805 of the cutting tool 802 and sleeve 804. FIG. 8B is an end view of the distal end of sleeve 804 and cutting tool 802 drawn in FIG. 8A.

### Disc Reconstruction Devices

Soft Tissue Implants

In one embodiment, as seen in FIG. 3K, disc reconstruction device 370 has an elongate portion 371 adapted to fit in the disc space between the surrounding vertebrae and an enlarged end portion 372 having a height that is larger than the distance between the vertebrae, such that at least a portion of the enlarged end portion extends to cover at least a portion of the surrounding vertebrae, i.e., the vertebrae cranial and caudal to the disc replacement device.

FIG. 4A is a lateral view of a disc reconstruction device 400 having elongate portion 403 adapted to fit in the disc space between the surrounding vertebrae and an enlarged end portion 401 having a height that is larger than the distance between the vertebrae, such that at least a portion of the enlarged end portion extends to cover at least a portion of the surrounding vertebrae, i.e., the vertebrae cranial and caudal to the disc replacement device. Disc reconstruction device also includes anti-adhesion patches or areas 402, 404 on the ends of enlarged end portion 401 and elongate portion 403, respectively. FIG. 4B is an axial cross section of disc reconstruction device 400 drawn in FIG. 4A. FIG. 4C is a lateral view of an alternative embodiment of disc reconstruction device 405, which is generally cylindrical in shape. Disc reconstruction device 405 may also include anti-adhesion patches or areas 406, 407 on the ends of the device to prevent the formation of adhesions.

FIG. 4D is an axial cross section of an alternative embodiment of a disc reconstruction device. Disc reconstruction device 408 has a plurality of extensions or fingers and has a larger surface area than disc reconstruction device 400 drawn in FIG. 4B. FIG. 4E is an axial cross section of disc reconstruction device 409 in the form of a sheet of mesh that has been folded and fastened to have a plurality of extensions or fingers that serve to increase the surface area of the device. The enlarged surface areas of the devices may facilitate tissue in-growth into the devices and increase the friction between the devices and the disc tissue. The devices may have additional surface features such as ribs, tines, barbs, teeth, etc. to increase the friction between the devices and the disc. The devices may be press fit into the space created in the disc.

FIG. 4F is an axial cross section of an alternative embodiment. Disc reconstruction device 412 has a lumen 410 therethrough. FIG. 4G is an axial cross section of an alternative embodiment in the form of hollow device 411 having lumen 413 therethrough. Hollow device 411 has a plurality of extensions of fingers that result in a larger surface area than device 412 drawn in FIG. 4F. Material, such as tissue from the defective disc, may be placed into the lumens of the devices before inserting the devices into the disc. Alternative synthetic or biologic materials, such as polymers, including but not limited to polyurethane or hydrogel, may be added to the devices.

Disc reconstruction devices are preferably made of materials that promote in-growth of disc tissue. For example, the disc reconstruction device may be made of polyester, polypropylene, or ePTFE mesh. The pores within the mesh are sized to optimize tissue in-growth. For example, the pores may be between about 10 and about 2000 microns, alternatively between about 100 and about 1500 microns, alternatively between about 250 and about 1000 microns. The anterior surface of the device could be covered with an ePTFE film to minimize adhesions to such structures as the esophagus. The ePTFE film could have pores as small 5 microns, alternatively as small as 4 microns, alternatively as small as 3 microns to minimize adhesion formation. A similar ePTFE film could be attached to the posterior portion of the device to minimize adhesions to the nerves or dura.

Alternatively, the disc reconstruction device may be constructed of biologic tissue, including allograft, autograft, or xenograft tissue. Examples of such biologic tissues include tendons, fascia, demineralized bone matrix, intestinal sub-mucosa, dermis, or other tissues from the body. As a further alternative, the disc reconstruction device may be made of a bioresorbable material, such as a collagen matrix, or combinations of bioresorbable materials and non-resorbable materials. The disc reconstruction device may also contain materials that allow visualization of the device during the procedure and after the procedure. The disc reconstruction device must also be MRI compatible. The disc reconstruction device could release therapeutic medical substances such as antibiotics, cytokines, or local anesthetics.

Bone and Soft Tissue implants

As described previously, FIGS. 9C and D illustrate implants that have both bone and soft tissue components. FIG. 9C is a lateral view of the embodiment of the invention drawn in FIG. 9B. Bone in-growth section 910 of the device may be made of titanium, ceramic, tantalum, or other material that promotes bone in-growth. The surface of bone in-growth section 910 of the device may be treated with hydroxyappetite, plasma spray titanium, or other treatment to promote bone in-growth. Bone in-growth section 910 may additionally have external threads that enable the device to be screwed into the vertebra. Alternatively, the external surface of bone in-growth component 910 may have teeth or other features to improve the press-fit of impacted devices. Soft tissue in-growth component 912 is preferably made of a porous mesh such as polypropylene, polyester, or ePFTE. Alternatively, soft tissue in-growth component 912 could be manufactured from allograft, autograft, or xenograft tissue. Tissue such as tendon, ligament, fascia, intestinal sub-mucosa, dermis, or other tissues could be used to facilitate soft tissue in-growth.

FIG. 9D is a sagittal cross section of an alternative embodiment of the invention. Mesh material 920, with excellent tissue in-growth characteristics, surrounds a device having bone in-growth section 910 and soft-tissue in-growth section. In one embodiment, the device may have a dowel shaped piece of bone 922 and disc component 924. Bone and disc components 922, 924 may be removed from the spine with a “hole saw” tool. The hole saw could be used in the method taught in FIG. 9A. The disc and/or bone filled device may be impacted into the hole in the spine in the method taught in FIG. 9B. Mesh component 922 holds disc tissue 924, improves the press fit between bone dowel 922 and the hole in the vertebra, and serves as a scaffold for cells to migrate from the spine and into the bone 922 and disc 924 tissue within mesh sleeve 920.

The anterior portion of mesh component 920 may have a flap 930. Flap or strap 930 helps hold the bone component within the spine. Mesh component 920 may be fastened to the spine with staples, helical tacks, screws, sutures, suture anchors, or other fastening methods and devices. The bone and disc dowel component 922, 924 may be placed into a container with circulating fluids. Oxygen and/or nutrients from the fluid could help preserve the cells in the tissue while the tissue is out of the body.

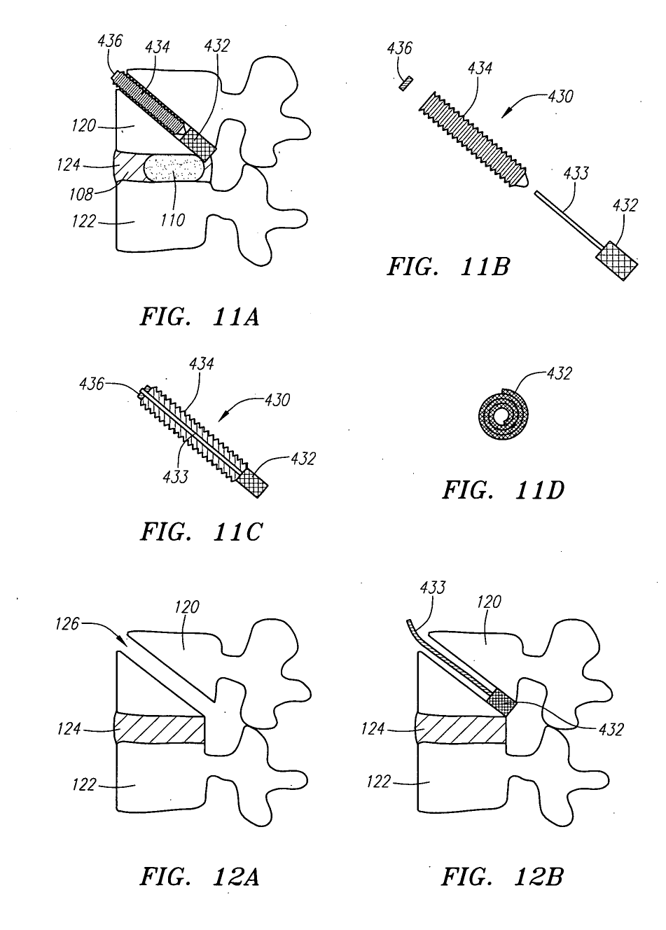

FIG. 11A is a lateral view of partial sagittal cross section of a portion of the spine and an alternative embodiment of the invention. Device 430 has been used to reconstruct the hole in vertebra 120 and the defective region of intervertebral disc 124. Device 430 extends into the spinal canal and anulus fibrosus 108 of the disc. Alternatively, the device could extend into the nucleus pulposus 110, the anulus fibrosus 108 and the nucleus pulposus 110, or neither the anulus fibrosus 108 nor the nucleus pulposus 110. For example, the device and the hole could be limited to the vertebra and or spinal canal without entering the disc. Embodiments of the invention that enter the disc preferably do not cross the disc and enter the adjacent vertebra.

FIG. 11B is an exploded lateral view of device 430 drawn in FIG. 11A. Device 430 includes a distal component that facilitates soft tissue in-growth 432, elongate member 433, elongate threaded member 434, and clamp 436. Soft tissue in-growth component 432 is preferably made of a porous mesh such as polypropylene, polyester, or ePFTE. Alternatively, soft tissue in-growth component 432 could be manufactured from allograft, autograft, or xenograft tissue. Tissue such as tendon, ligament, fascia, intestinal sub-mucosa, dermis, or other tissues could be used to facilitate soft tissue in-growth. Soft tissue in-growth component 432 is preferably about 1 to about 7 mm in diameter and about 1 to about 7 mm in length. Alternatively, soft tissue in-growth component 432 could be about 8 mm, alternatively about 9 mm, or alternatively about 10 mm or more in diameter and/or length. Elongate member 433, which can be a flexible, suture or cord-like component, extends from the proximal end of soft tissue in-growth component 432. Elongate member 433 is preferably about 1-2 mm in diameter and about 5 to 30 mm in length. Alternatively, elongate member 433 could be about 3 mm, alternatively about 4 mm, or alternatively about 5 mm or more in diameter. Additionally, elongate member 433 could be about 10 to about 25 mm, alternatively about 15 to about 20 mm in length. Elongate threaded member 434 is preferably cannulated and is passed over elongate member 433 and screwed into the hole drilled into vertebra 120. Clamp 436 is used to connect elongate threaded member 434 and soft tissue in-growth component 432. Elongate threaded member 434 is preferably made of MRI compatible resorbable material. Suitable bio-resorbable materials include polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid). Alternatively, the elongate threaded member could be made of bone (including allograft or xenograft bone), titanium, stainless steel, plastic (including Delrin, polyethylene, or other polymer), bio-active cements or other in-situ curing or fully cured material. Elongate threaded member 434 is preferably about 2 to about 9 mm in diameter, alternatively about 3 to about 8 mm in diameter, alternatively about 4 to about 7 mm in diameter. Elongate threaded member 434 is preferably about 3 mm to about 15 mm in length, alternatively about 5 mm to about 12 mm in length, alternatively about 7 mm to about 10 mm in length. Clamp component 436 is fastened to the proximal end of elongate member 433. Clamp 436 may be crimped over or otherwise fastened to elongate member 433. Alternatively, clamp 436 may be made of a shape memory material that grasps elongate member 433. Clamp 436 is preferably 2 to 5 mm in length and 1 to 4 mm in diameter. Alternatively, clamp 436 may be more than about 5 mm in length and more than about 5 mm in diameter. FIG. 11C is a longitudinal cross section of the device 430.

FIG. 11D is an end view of the distal end of device 430. In this embodiment, the in-growth material in soft tissue component 432 is coiled. This configuration increases the stiffness of the component. Alternatively, the in-growth component could be manufactured in a plug-like shape.

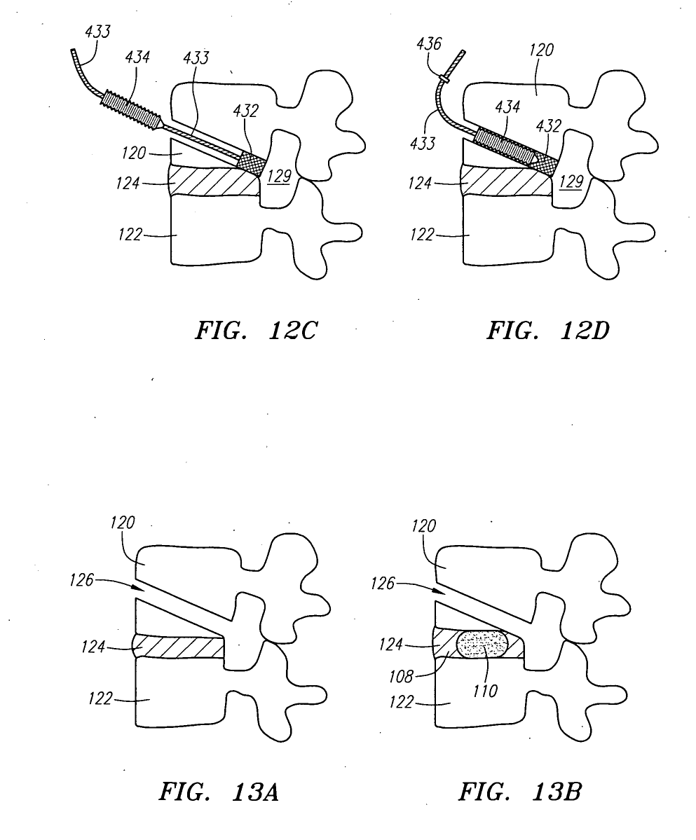

FIG. 12A is a lateral view of a partial sagittal cross section of the spine. Hole or tunnel 126 has been drilled through vertebra 120. Hole or tunnel 126 extends through vertebra 120 and ends in the area of spinal pathology on the posterior side of the vertebra and disc. Robotic and/or navigational tools, as described previously, could be used to make the tunnel and/or determine the location of the tunnel. Instruments may be placed through tunnel 126 to remove herniated nucleus pulposus, osteophytes, tumors, or other spinal pathology.

FIG. 12B is a lateral view of a partial sagittal cross section of the spine and the embodiment of the invention drawn in FIG. 12A. Soft tissue in-growth component 432, with attached elongate member 433 extending proximally, has been placed into hole 126 in the spine.

FIG. 12C is a lateral view of a partial sagittal cross section of the spine and the embodiment of the invention drawn in FIG. 12B. Elongate threaded member 434 was placed over elongate member 433 of the in-growth component.

FIG. 12D is a lateral view of a partial sagittal cross section of the spine and the embodiment of the invention drawn in FIG. 12C. Elongate threaded member 434 has been advanced into vertebra 120. Clamp 436 is advanced over elongate member 433. Clamp 436 is placed against elongate threaded member 434 and fastened to elongate member 433. Tension is applied to the elongate member 433 before fastening clamp 436. The portion of elongate member 433 proximal to clamp 434 is cut and removed.

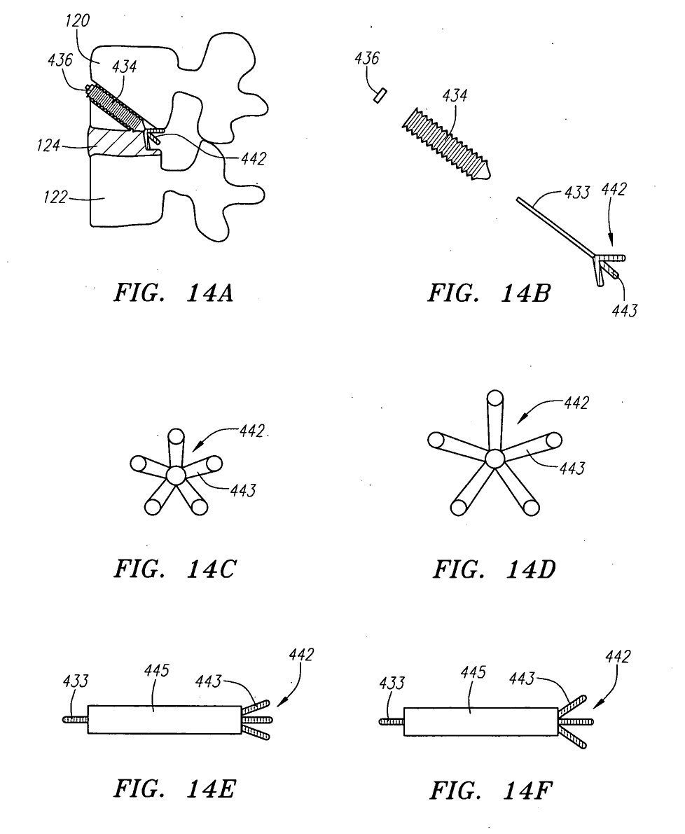

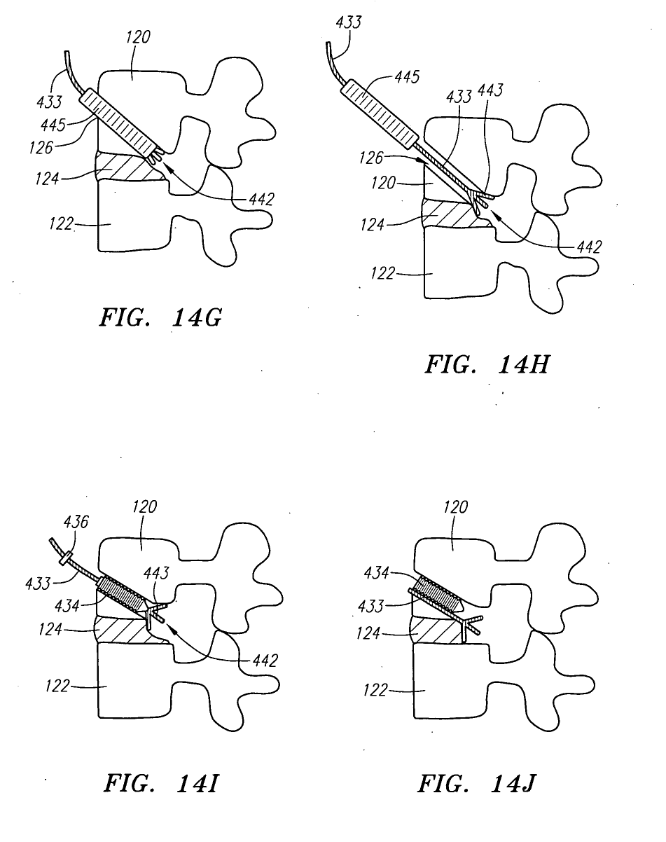

FIG. 14A is a lateral view of a partial sagittal cross section of the spine and an alternative embodiment of the invention having a distal end that radially expands. FIG. 14B is an exploded lateral view of the embodiment of the invention drawn in FIG. 14A. Elongate threaded member 434 and clamp 436, as described in the text of FIG. 11B, are placed over an elastic or super elastic component comprising elongate member 433 and expandable distal end 434, which expands in a radial direction. The elastic component may be made of material with spring-like elastic properties and/or shape memory materials. These materials include Nitinol, titanium, plastic, or other elastic and/or shape materials. The elastic component preferably has dimensions similar to the soft-tissue in-growth component described in the text of FIG. 11B.

FIG. 14C is a view of distal end 442 of the elastic/shape memory component drawn in FIG. 14B, which has a plurality of appendages or projections 443 that expand radially. For example the distal end 442 could have 2, alternatively 3, alternatively 4, alternatively 5, alternatively 6, alternatively 7, alternatively 8, alternatively 9, alternatively 10, alternatively 11, alternatively 12, alternatively 13 or more appendages or projections 443.

FIG. 14D is a view of the distal end 442 of the elastic/shape memory component drawn in FIG. 14C. Distal end 442 of the elastic component is drawn in its expanded configuration. Distal end 442 of the component may change from its contracted shape to its expanded shape by releasing compression on the side of the component or in reaction to the environment surrounding the component. For example, the end of the device may expand as the component is exposed to the patient's body temperature. Alternatively, a sleeve may be used to hold the elastic/shape memory component in its contracted shape.

FIG. 14E is a lateral view of the elastic/shape memory component drawn in FIG. 14B and a sleeve. The sleeve may be used to hold the elastic/shape memory component in its contracted shape. Distal end 442 may be maintained in its contracted shape by sleeve 445 wherein movement of sleeve 445 proximally (or movement of distal end 442 distally) such that distal end 442 is no longer located within a lumen of sleeve 445 will result in expansion of projections 443.

FIG. 14F is a lateral view of the embodiment of the invention drawn in FIG. 14E. Distal end 442 of the elastic/shape memory component expands as sleeve 445 is retracted.

FIG. 14G is a lateral view of a partial sagittal cross section the spine and the embodiment of the invention drawn in FIG. 14A. Sleeve 445 and elastic/shape memory component are placed into tunnel 126 through the vertebra. Distal end 442 of the elastic component is maintained in its contracted shape by sleeve 445.

FIG. 14H is a lateral view of a partial sagittal cross section of the spine and the embodiment of the invention drawn in FIG. 14G. Sleeve 445 has been retracted. Arms 443 of the elastic/shape memory component, which have been positioned adjacent to disc 124, expand as sleeve 445 is retracted and/or the component reacts to its environment. Arms 443 expand into disc 124. Arms 443 of the component prevent extrusion of disc fragments from disc 124. Expansion of arms 443 of the elastic/shape memory component opposite intervertebral disc 124 is limited by the wall of tunnel 126.

FIG. 14I is a lateral view of a partial sagittal cross section of the spine and the embodiment of the invention drawn in FIG. 14H. Elongate threaded member 434 has been advanced over elongate member 433. Tension on elongate member 433 during insertion of elongate threaded member 434 prevents inadvertent advancement of expandable distal end 422 into the spinal canal. Elongate threaded member 434 and elongate member 433 can be fastened together with clamp 436. Clamp 436 and elastic/shape memory component could be connected with shape memory fastening technology. Alternatively, the elongate threaded member and the elastic/shape memory component could be connected with shape memory technology. For example, the elongate threaded member could contract or the elastic/shape memory component could expand, or both, to fasten the components together.

FIG. 14J is a lateral view of a partial sagittal cross section of the spine and an alternative embodiment of the invention drawn in FIG. 14A. The elastic/shape memory component comprising elongate member 433 and expandable distal end 442 having projections 443 is held in the spine by elongate threaded member 433 placed adjacent to elongate member 433. Elongate threaded member 434 forces the elongate member 433 against the wall of tunnel 126. The configuration eliminates the need for the clamp component and forces the distal end of the elastic/shape memory component towards disc 124. Elongate member 433 is cut and removed after placement of the elongate threaded member. Alternatively, the elastic/shape memory component could be released from an insertion tool after placement of the elongate threaded member.

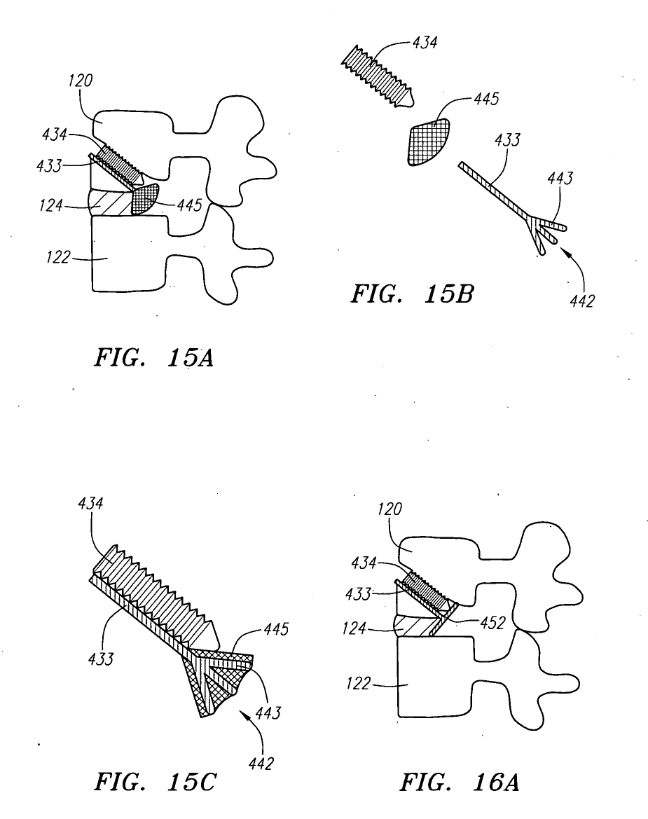

FIG. 15A is a lateral view of a partial sagittal cross section of the spine having an alternative implant positioned in the vertebra and disc having a covering over the expandable distal end. Soft tissue in-growth component 445 has been placed over projections 443 of distal end 442 of the elastic/shape memory component in a manner similar to material over the metal arms of an umbrella. The soft tissue in-growth materials described in the text with respect to FIG. 11B could be used to cover the elastic/shape memory component. These include a porous mesh such as polypropylene, polyester, or ePFTE. Alternatively, the soft tissue in-growth component could be manufactured from allograft, autograft, or xenograft tissue. Tendon, ligament, fascia, intestinal sub-mucosa, dermis, or other tissues could be used to facilitate soft tissue in-growth. As seen in FIG. 14J, elongate threaded member 433 has been placed adjacent to elongate member 433, thereby forcing elongate member 433 against the wall of tunnel 126. As discussed previously, this configuration eliminates the need for the clamp component and forces the distal end of the elastic/shape memory component towards disc 124. FIG. 15B is an exploded lateral view of the embodiment of the invention drawn in FIG. 15A. FIG. 15C is a lateral view of a longitudinal cross section of the embodiment of the invention drawn in FIG. 15A.

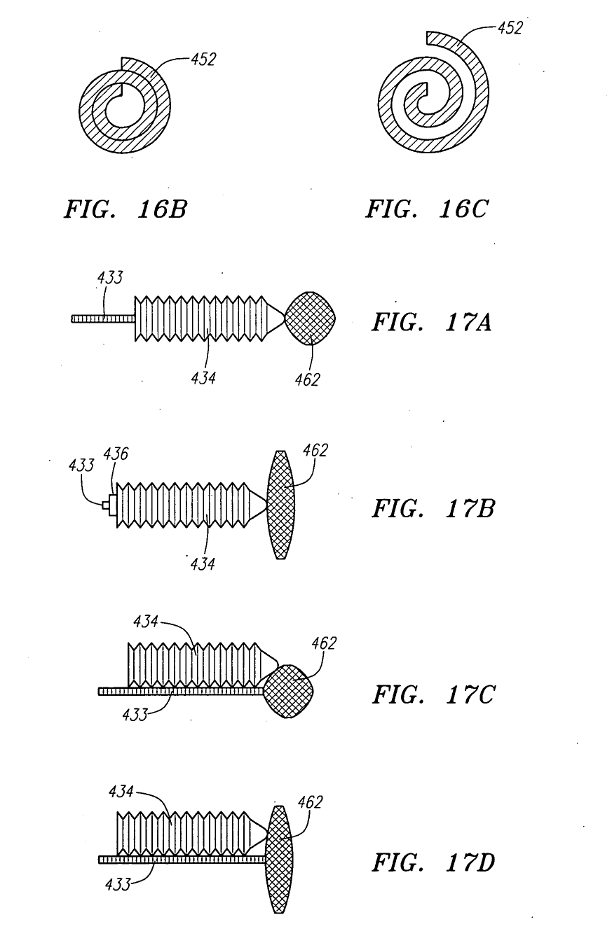

FIG. 16A is a lateral view of a partial sagittal cross section of the spine having an alternative implant positioned in the vertebra and disc, wherein the distal end has an expandable spiral or coil. Expandable distal end 452 has a coiled or spiral shape that is tightly wound in its contracted configuration (see FIG. 16B) and more loosely wound in its expanded configuration (see FIG. 16C). Expandable distal end 452 expands in a radial direction after passage through the tunnel in the vertebra. The coiled or spiraled distal end 452 can be made from a material with spring-like elastic properties and/or shape memory materials such as Nitinol, titanium, plastic, or other elastic and/or shape materials.

FIG. 17A is a lateral view of an alternative embodiment of the invention having an alternative implant positioned in the vertebra and disc, wherein the distal end can expand radially. FIG. 17B is a lateral view of the embodiment of the invention drawn in FIG. 17A with expandable distal end 462 of the soft tissue in-growth component drawn in its expanded configuration. Expandable distal end 462 includes an expandable metal or plastic frame, similar to a malecot, having a covering to promote tissue in-growth. The expandable frame may have a tensioning or actuation member (not shown) attached to its distal end. Movement of the tensioning or actuation member proximally forces the distal end 462 of the in growth component against elongate threaded member 434, thereby radially expanding distal end 462. Alternatively, the expandable frame could be made of shape memory materials that cause the distal end to expand in reaction to a patient's body temperature. Clamp 436 may be placed over elongate member 433 to maintain tension on the device.

FIG. 17C is a lateral view of an alternative embodiment of the invention drawn in FIG. 17A that does not require a clamp when placed within a tunnel in the vertebra. The device is fastened to the spine by an interference fit between the elongate member 433 and elongate threaded member 434. FIG. 17D is a lateral view of the embodiment of the invention drawn in FIG. 17C, with distal end 462 drawn in its expanded configuration. Shape memory materials could be used to cause the device to expand in reaction to a patient's body temperature. Alternatively, distal end 462 could expand as elongate threaded member 434 is advanced against the side of distal end 462. Tension on elongate member 433 during placement of threaded member 434 prevents inadvertent advancement of elongate member 433 or expandable distal end 462. The portion of elongate member 433 that protrudes from the vertebra after placement of threaded member 434 is cut and removed at the end of the procedure.

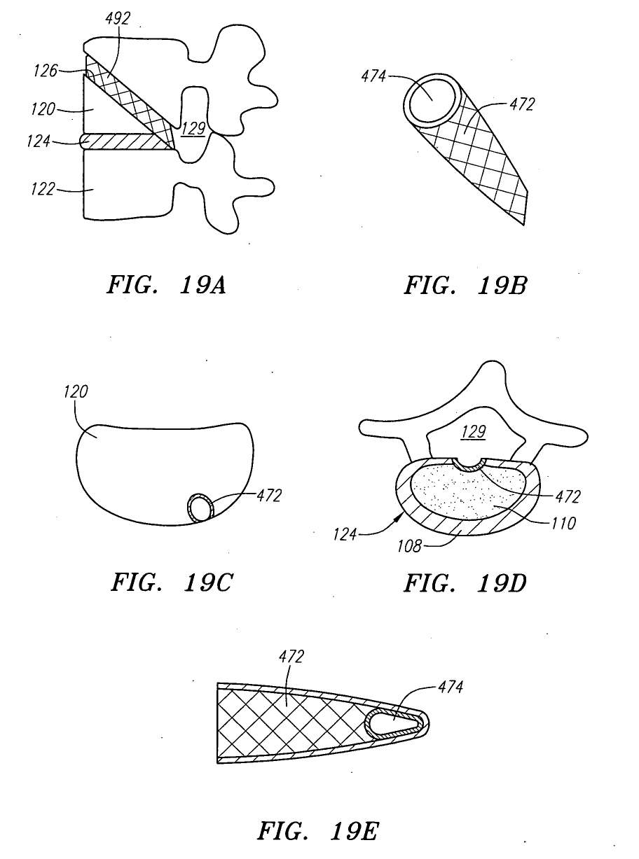

FIG. 19A is a lateral view of a partial sagittal cross section of the spine and an alternative embodiment. Device 472 has been placed into tunnel 126 in vertebra 120 and disc 124. FIG. 19B is an oblique view of the proximal end of the device drawn in FIG. 19A. Device 472 is porous and preferably made of a shape memory material such as nitinol. Device 472 preferably has lumen 474 and a tapered distal end, wherein when the device is properly positioned in the vertebra and disc, it will not extend into spinal canal 129. Bone from the vertebra and fibrous tissue from the disc can grow through the device. Furthermore, device 472 may have teeth or spikes over its exterior. The teeth or spikes would become embedded into the vertebra and/or the disc. The device can be filled with BMP soaked collagen sponges such as Infuse (Medtronic Sofamor Danek, Memphis Tenn.) or OP-1 (Stryker, Kalamazoo, Mich.), Demineralized bone matrix, or other material that facilitates bone growth and/or soft tissue growth.

Device 472 expands in a radial direction after placement in tunnel 126. Expansion may occur secondary to environment change, such as warming of the device by a patient's body heat. Expansion may alternatively be aided by inflation of a balloon within the device. In such an embodiment, the balloon and catheter would be withdrawn after expanding the device.

FIG. 19C is an axial cross section of cranial vertebra 120 with device 472 located within the vertebra. FIG. 19D is an axial cross section of disc 124 and device 472, which lies along the edge of the surgically treated disc 124. Device 124 does not extend into spinal canal 129. Device 472 prevent disc material from extruding through the defect in the disc and into the spinal canal. FIG. 19E is an oblique view of the distal end of device 472. The portion of device 472 that lies adjacent to the disc may be covered with a material that promotes tissue in-growth. Materials such as polyester, polypropylene, ePTFE, or similar material may be used for the in-growth component.

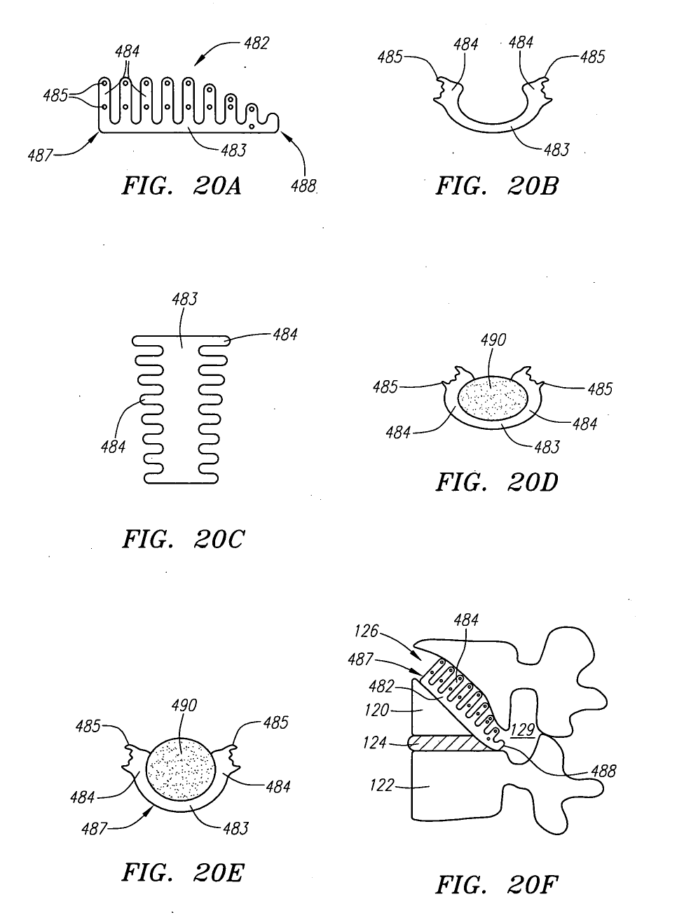

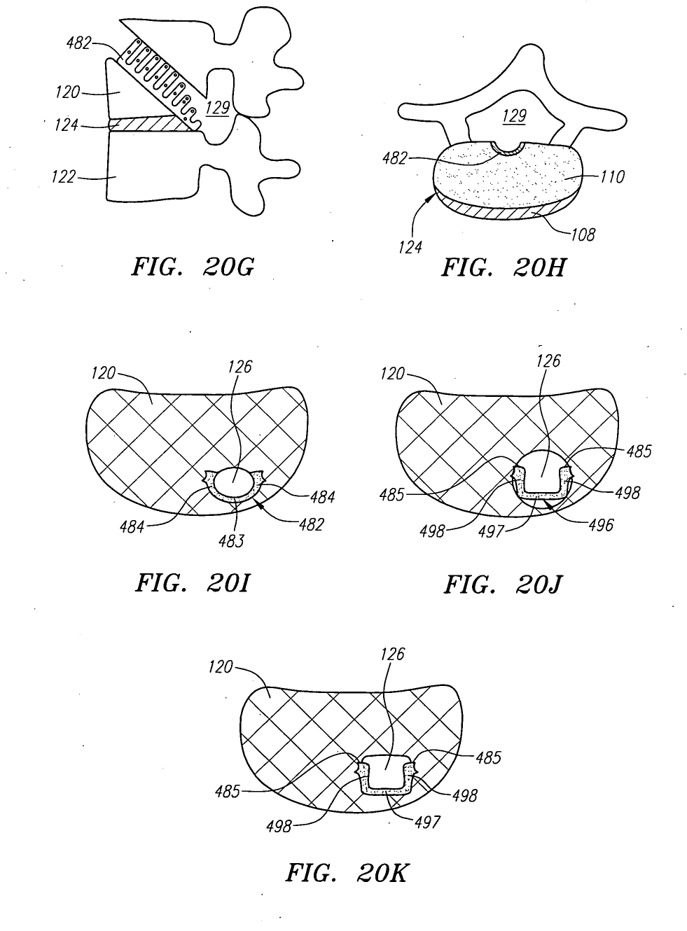

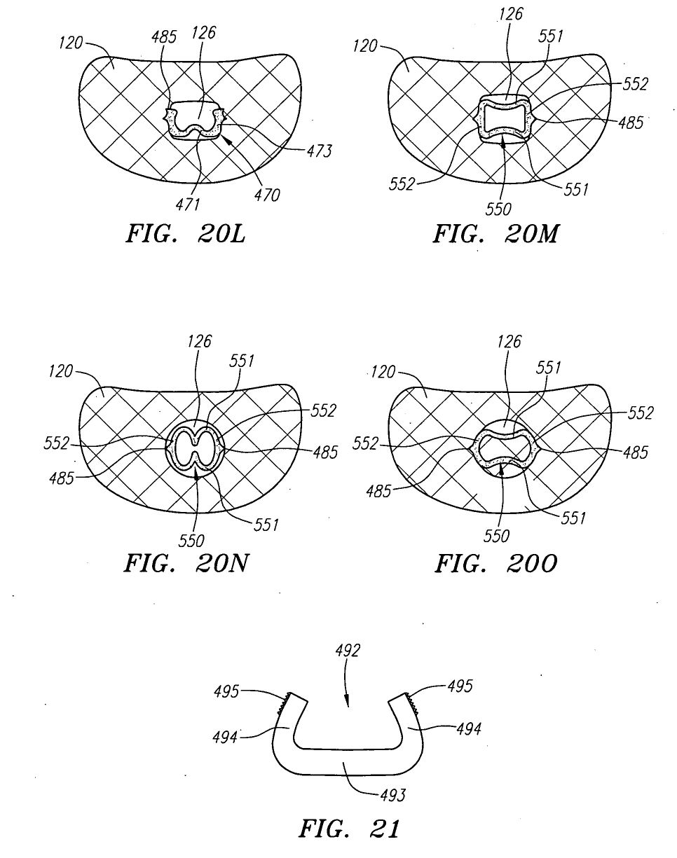

FIG. 20A is a lateral view of an alternative embodiment of the invention that can be inserted into a vertebra and a disc. FIG. 20B is a view of the proximal end of the embodiment of the invention drawn in FIG. 20A. FIG. 20C is a view of the inferior surface of the embodiment of the invention drawn in FIG. 20A. As depicted, device 482 has a U-shaped cross section and has spine (or generally rectangular member) 483 having a first and second longitudinal side and a plurality of ribs 484 extending from the first and second longitudinal edges. The length of ribs 484 may be different lengths. For example, the ribs at distal end 488 may be longer than the ribs at proximal end 487. Alternatively, the ribs at distal end 488 may be shorter than the ribs at proximal end 487. The tips of ribs 484 may, although not necessarily, have teeth, tines, or other projections 485. Device 482 may have shapes other than a U-shaped cross section. For example, the device could have a generally circular, oval, triangle, rectangle, or other shape on cross section. Devices with non-circular cross sections resist rotation about the longitudinal axis of the device. Similarly, tunnels with non-circular cross sections minimize the risk of devices rotating about their longitudinal axes. Such rotation may rotate the distal tip of the device away from the defective region of the disc.

It is preferably supplied to physicians in various sizes and possibly different shapes. For example, the device could be supplied with a length of about 5 mm, alternatively about 6 mm, alternatively about 7 mm, alternatively about 8 mm, alternatively about 9 mm, alternatively about 10 mm, alternatively about 12 mm, alternatively about 14 mm, alternatively about 16 mm, alternatively about 18 mm, alternatively about 20 mm, alternatively about 22 mm, alternatively about 24 mm, alternatively about 26 mm, alternatively about 28 mm or more. The device could similarly be supplied with a width of about 3 mm, alternatively about 4 mm, alternatively about 5 mm, alternatively about 6 mm, alternatively about 7 mm, alternatively about 8 mm, alternatively about 9 mm, alternatively about 10 mm, alternatively about 12 mm, alternatively about 14 mm, alternatively about 15 mm or more.

The device is expandable from a contracted state to an expanded state. A distance between a free end of a rib on the first longitudinal edge and a free end of a corresponding rib on the second longitudinal edge is smaller in the contracted state than in the expanded state. Additionally, the generally rectangular member and the plurality of ribs define a volume. The defined volume is smaller in the contracted state than in the expanded state. The defined volume in the contracted state may be about 10% less than the defined volume in the expanded state, alternatively 15% less, alternatively 20% less, alternatively 25% less, alternatively 30% less, alternatively 40% less, alternatively 50% less.

The device is preferably made of an elastic or super-elastic shape memory material such as nitinol. Ribs 484 expand outward in a reaction to an environment change, such as an increase in temperature when the device is placed into a patient's body. The sides of device 482 are preferably parallel. In another embodiment, device 482 may be truncated with the proximal end of the device being wider or narrower than the distal end of the device.

The device may be expanded by inflating a balloon within the device. FIG. 20D is a view of proximal end 488 of the embodiment of the invention drawn in FIG. 20B and balloon 490. Balloon 490 is preferably connected to an inflation device. A catheter may be used to connect the balloon to the inflation device. Balloon 490 is drawn in its deflated configuration and device 482 is drawn in its contracted configuration. FIG. 20E is a view of the proximal end of the embodiment of the invention drawn in FIG. 20D and balloon 490. Balloon 490 has been inflated, thereby expanding device 482 into its expanded configuration having opposing ribs substantially parallel. Alternative methods or devices may be used to expand device 482. For example, a screw could be advanced along a longitudinal axis of spine 483, between the plurality of ribs extending from the first and second longitudinal edges of the spine. Similar to the balloon, the screw could be used to increase the distance between the tips of ribs 484 and force spine 483 against the wall of tunnel 126. The screw could optionally be left inside the tunnel.

FIG. 20F is a lateral view of a sagittal cross section through a portion of the patient's spine and a lateral view of the embodiment of the invention drawn in FIG. 20A. Device 482 has been inserted into tunnel 126 milled into the patient's spine. Device 482 expanded within tunnel 126, either in reaction to an increase in temperature or as a result of an inflated balloon. The device preferably bends slightly at the caudal vertebral endplate of cranial vertebra 120. The distal tip 488 of device 482 preferably contacts the cranial vertebral endplate of caudal vertebra 122. FIG. 20F acts as a scaffold to facilitate bone growth into and through the tunnel in the vertebra. Bone growth into the tunnel closes the tunnel. The sides of tunnel 126 may be milled parallel to one another. Alternatively, the sides of tunnel 126 may taper. The proximal end of the tunnel may be wider than the distal end of the tunnel. Such a shape increases the angle at which instruments may be directed towards the spinal canal. Conversely, the distal end of the tunnel may be wider than the proximal end of the tunnel, which increases the angle at which instruments may be directed towards the spinal canal, preserves vertebral bone, and may facilitate removal of bone spurs or disc fragments.

FIG. 20G is a lateral view of a partial sagittal cross section of a portion of the spine and a lateral view of the embodiment of the invention drawn in FIG. 20F with the spine drawn in a flexed position. Flexion increases the height of the posterior portion of the disc and increases the risk of recurrent herniated nucleus pulposus. As depicted in FIG. 20G, device 482 extends to remain adjacent to the cranial vertebral endplate of caudal vertebra 122 while in the flexed position. This reduces the risk of disc fragment extrusion around the tip of the device. FIG. 20I is an axial cross section of cranial vertebra 120 and the device drawn in FIG. 20G.

FIG. 20H is an axial cross section through a disc and the embodiment of the invention drawn in FIG. 20G. Distal tip 488 of device 482 surrounds the defective region of the disc. Distal tip 488 may lie within anulus fibrosus 108 or may lie within anulus fibrosus 108 and nucleus pulposus 110. Generally or alternatively, distal tip 488 of the device lies within the posterior 3 to 6 mm of disc 124. Alternatively, distal tip 488 of the device may lie within about 1 mm, alternatively within about 2 mm, alternatively within about 7 mm, alternatively within about 8 mm, alternatively within about 9 mm, alternatively within about 10 mm or more of spinal canal 129.

FIG. 20J is an axial cross section of cranial vertebra 120 and an alternative embodiment of the device. Device 496 has spine (or generally rectangular member) 497 and a plurality of ribs 498 extending from the longitudinal sides of spine 497. Depending on the shape of spine 497, device 496 may have a U-shaped cross section. Alternatively, where spine 497 is substantially straight or flat, ribs 497 may be substantially perpendicular to a plane defined by spine 497. As seen in FIG. 20J, where device 496 has been inserted into a generally circular or rounded tunnel 126 in vertebra 120, ribs 498 engage the vertebral surface of tunnel 126 but spine 497 does not substantially contact the sides of tunnel 126.

FIG. 20K is an axial cross section of cranial vertebra 120 having tunnel 126 with a different cross section. In tunnel 126 having a rectangular cross section, both spine 497 and ribs 498 contact and engage the vertebral surface of tunnel 16 and device 496 is not able to rotate about its longitudinal axis. This configuration prevents the device from rotating away from the defective region of the AF.

FIG. 20L is an axial cross section of cranial vertebra 120 and an alternative embodiment of the invention. Device 470 has a contracted shape for insertion into the tunnel and an expanded operational shape. In the contracted configuration, spine (or generally rectangular member) 471 of device 470 is curved (or non-linear). Straightening or flattening of spine 471 results in the widening of a width of the spine, which increases the friction fit between ribs 473 and spine 471 and the surrounding walls of tunnel 126. Projections 485 on the outside surface of ribs 473 also aid in the frictional engagement of the vertebral surface. Spine (or generally rectangular member) 471 of the device may be made from a shape memory material and may widen in reaction to a patient's body temperature.

FIG. 20M is an axial cross section of cranial vertebra 120 having tunnel 126 with a rectangular cross-section and an alternative embodiment of the invention. Device 550 has a contracted shape for insertion into the tunnel and an expanded operational shape. Device 550 has curved spines (or generally rectangular members) 551 across the top and bottom of the device, which are connected by ribs 552. As explained with respect to FIG. 20L, both spines 551 widen as the curves of the spines straighten in reaction to a patient's body temperature. As spines 551 straighten and widen, ribs 552 are forced outward against and possibly into the walls of tunnel 126, such that ribs 552 frictionally engage the vertebral surface of the tunnel. Projections 485 on the outside surface of ribs 552 also aid in the frictional engagement of the vertebral surface.

FIG. 20N is an axial cross section of cranial vertebra 120 having tunnel 126 with a circular cross-section and device 550 depicted in FIG. 20M. Device 550 is drawn in its contracted first shape, wherein spines 551 are curved (or non-linear). This contracted shape facilitates insertion of the device into tunnel 126. Device 550 may expand in reaction to a patient's body temperature to assume the second, expanded, shape drawn in FIG. 20O. Although some portion of spines 551 may not contact the tunnel walls, spines 551 have straightened (are less curved) and ribs 551 are frictionally engaging the vertebral surface of tunnel 126.

As with the device with a single spine, device 550 is expandable from a contracted state to an expanded state. A distance between a distance between a midpoint of a rib connecting the first longitudinal edges and a mid-point of a corresponding rib connecting the second longitudinal edges is smaller in the contracted state than in the expanded state. Additionally, the first and second spines (generally rectangular members) and the plurality of ribs define a volume. The defined volume is smaller in the contracted state than in the expanded state. The defined volume in the contracted state may be about 10% less than the defined volume in the expanded state, alternatively 15% less, alternatively 20% less, alternatively 25% less, alternatively 30% less, alternatively 40% less, alternatively 50% less.

FIG. 21 is a view of the proximal end of an alternative embodiment of the invention drawn in FIG. 20A. Spine 493 of device 492 is thicker than ribs 494 of the device. Additionally, ribs 494 have a plurality of teeth 495 located on the outer portion of the distal region of the ribs. The number of teeth have increased but became smaller than the teeth drawn in FIG. 20B. Alternatively, the teeth could be eliminated. Device 492 can be made of the same materials as described with respect to FIGS. 20A-I. Similarly, device 492 could be deployed in the same manner as described with respect to FIGS. 20A-I.

Although the foregoing invention has, for the purposes of clarity and understanding, been described in some detail by way of illustration and example, it will be obvious that certain changes and modifications may be practiced which will still fall within the scope of the appended claims.

1. A method of repairing a disc having herniated nucleus pulposus, wherein the disc is situated between a cranial and caudal vertebra, comprising the steps of:
inserting a guide wire into a disc using an anterior lateral approach to create a path;
removing at least a portion of the herniated nucleus pulposus;
attaching a suture to at least one of the cranial and caudal vertebra;
advancing a disc reconstruction device along the path into the disc; and
fastening the disc reconstruction device to at least one of the cranial and caudal vertebra with the suture.

2-25. (canceled)

26. A method for repairing a disc having a herniated nucleus pulposus, wherein the disc is situated between cranial and caudal vertebra, comprising the steps of:
forming a passage in the cranial or caudal vertebra that extends into the disc using an angled anterior approach that enters the cranial or caudal vertebra and terminates at a posterior region of the disc that includes the herniated nucleus pulposus;
removing at least a portion of the herniated nucleus pulposus; and
implanting a disc reconstruction device into the passage, wherein the disc reconstruction device has a bone in-growth component and a tissue in-growth component, and wherein the bone in-growth component is located in the cranial or caudal vertebra and the tissue in-growth component is located in the disc.

27-41. (canceled)

42. A method for removing a bone spur from a vertebra, comprising the steps of:
inserting a guide wire into a disc located adjacent to the vertebra using an anterior lateral approach to create a path;
advancing a cutting tool to the bone spur along the path;
removing at least a portion of the bone spur from the vertebra using the cutting tool;
advancing a disc reconstruction device along the path into the disc; and
fastening the disc reconstruction device to at least one of the disc or the vertebra.

43-56. (canceled)

57. A medical device for implantation into a spine of a patient, comprising:
a soft tissue in-growth component made from a material selected from the group consisting of porous mesh, allograft tissue, autograft tissue, xenograft tissue, and combinations thereof; and
a hard tissue in-growth component associated with the soft tissue in-growth component, and wherein the hard tissue in-growth component is made from a material selected from the group consisting of titanium, ceramic, and tantalum.

58-60. (canceled)

61. A medical device for implantation into a spine of a patient, comprising:
a mesh sleeve having a chamber and a closed distal end,
a soft tissue in-growth component comprising at least one of anulus fibrosus or nucleus pulposus obtained from the patient, wherein the soft tissue in-growth component is located in the chamber of the mesh sleeve; and
a hard tissue in-growth component comprising bone obtained from the patient, wherein the hard tissue in-growth component is located in the chamber of the mesh sleeve and is located proximal of the soft tissue in-growth component.

62-64. (canceled)

65. A medical device for implantation into a spine of a patient, comprising:
a soft tissue in-growth component comprising porous mesh;
an elongate member extending proximally from the soft tissue in-growth component;
an elongate threaded member having a lumen adapted to receive the elongate member; and
a fastener releasably attached at a distal end of the elongate member.

66-75. (canceled)

76. A medical device for implantation into a spine of a patient, comprising:
a soft tissue in-growth component comprising porous mesh;
an elongate member extending proximally from the soft tissue in-growth component; and
an elongate threaded member adapted to frictionally engage the elongate member at a position along a circumference of the elongate threaded member.

77-85. (canceled)

86. A medical device for implantation into a spine of a patient, comprising:
an expandable implant composed of an elastic or shape-memory material;
an elongate member extending proximally from the expandable implant; and
an elongate threaded member having a lumen adapted to receive the elongate member; and
a fastener releasably attached at a distal end of the elongate member.

87-94. (canceled)

95. A medical device for implantation into a spine of a patient, comprising:
an expandable implant composed of an elastic or shape-memory material;
an elongate member extending proximally from the expandable implant; and
an elongate threaded member adapted to frictionally engage the elongate member at a position along a circumference of the elongate threaded member.

96-99. (canceled)

100. A medical device for implantation into a spine of a patient, comprising:
an elongate tubular structure having a proximal end, a distal tapered end, and a lumen therebetween,
wherein the elongate tubular structure is composed of a porous mesh made from a shape memory metal.

101-102. (canceled)

103. A method for repairing a disc having a herniated nucleus pulposus, wherein the disc is situated between a cranial and caudal vertebra, comprising the steps of:
forming a passage in the cranial vertebra that extends into the disc using an angled anterior approach that enters the cranial vertebra and terminates at a posterior region of the disc that includes the herniated nucleus pulposus;
removing a portion of the herniated nucleus pulposus;
locating an elongate tubular structure in the passage, wherein the elongate tubular structure has a proximal end, a distal tapered end, and a lumen therebetween, and wherein the elongate tubular structure is composed of a porous mesh; and
expanding the elongate tubular structure to frictionally engage the vertebral surface of the passage.

104-107. (canceled)

108. A medical device for implantation into a spine of a patient, comprising:
a generally rectangular member having first and second longitudinal edges; and
a plurality of ribs extending from the first and second longitudinal edges,
wherein the generally rectangular member and plurality of ribs are composed of an elastic or shape-memory material, and wherein the device is expandable from a contracted state to an expanded state.

109. The device of claim 108, wherein each of the plurality of ribs further comprises at least one projection at a free end of each of the plurality of ribs.

110. The device of claim 109, wherein the projection is a tine or barb.

111. The device of claim 108, wherein the plurality of ribs are composed of nitinol.

112. The device of claim 108, wherein the generally rectangular member and each of the plurality of ribs each have a thickness, and wherein the thickness of the generally rectangular member is larger than the thickness of each of the plurality of ribs.

113. The device of claim 108, wherein a distance between a free end of a rib on the first longitudinal edge and a free end of a corresponding rib on the second longitudinal edge is smaller in the contracted state than in the expanded state.

114. The device of claim 108, wherein the generally rectangular member and the plurality of ribs define a volume, and wherein the defined volume is smaller in the contracted state than in the expanded state.

115. The device of claim 108, wherein the plurality of ribs are expandable from the contracted state to the expanded state.

116. The device of claim 108, wherein the generally rectangular member is expandable from the contracted state to the expanded state.

117. The device of claim 116, wherein the generally rectangular member is curved in the contracted state.

118. The device of claim 108, further comprising an additional generally rectangular member having first and second longitudinal edges, and wherein the plurality of ribs connect the first and second longitudinal edges of the generally rectangular member and the additional generally rectangular member.

119. The device of claim 118, wherein the additional generally rectangular member is curved.

120. A method for repairing a disc having herniated nucleus pulposus, wherein the disc is situated between a cranial and caudal vertebra, comprising the steps of:
forming a passage in the cranial vertebra that extends into the disc using an angled anterior approach that enters the cranial vertebra and terminates at a posterior region of the disc that includes the herniated nucleus pulposus;
placing a device having a generally rectangular member having first and second longitudinal edges and a plurality of ribs extending from the first and second longitudinal edges into the passage; and
expanding the device to frictionally engage the vertebral surface of the passage.

121. The method of claim 120, wherein the device comprises a shape memory material and wherein the device expands as a result of a temperature change after it is located in the passage.

122. The method of claim 120, wherein the device expands as a result of expanding a balloon located between the plurality of ribs and along a longitudinal axis of the generally rectangular member.

123. The method of claim 120, wherein the passage has a circular cross-section.

124. The method of claim 120, wherein the passage has a rectangular cross-section.

125. The method of claim 120, wherein the generally rectangular member is curved and wherein the device expands by straightening the generally rectangular member.

126. The method of claim 125, wherein the plurality of ribs frictionally engage the vertebral surface as the generally rectangular member straightens.

127. The method of claim 125, wherein the generally rectangular member frictionally engage the vertebral surface as the generally rectangular member straightens.

128. The method of claim 120, wherein the device is placed into the passage using a visualization technique selected from the group consisting of MRI, CT, and fluoroscopy

129. A medical device for implantation into a spine of a patient, comprising:
first and second generally rectangular members each having first and second longitudinal edges; and
a plurality of ribs extending between the first and second longitudinal edges of the first and second generally rectangular members,
wherein the first and second generally rectangular members and the plurality of ribs are composed of an elastic or shape-memory material, and wherein the device is expandable from a contracted state to an expanded state.

130-138. (canceled)

139. A method for repairing a disc having herniated nucleus pulposus, wherein the disc is situated between a cranial and caudal vertebra, comprising the steps of:
forming a passage in the cranial vertebra that extends into the disc using an angled anterior approach that enters the cranial vertebra and terminates at a posterior region of the disc that includes the herniated nucleus pulposus;
placing a device into the passage having first and second generally rectangular members each having first and second longitudinal edges and a plurality of ribs extending between the first and second longitudinal edges of the first and second generally rectangular members; and
expanding the device to frictionally engage the vertebral surface of the passage.

140-147. (canceled)

148. A method for repairing a disc having a herniated nucleus pulposus, wherein the disc is situated between a cranial and caudal vertebra, comprising the steps of:
forming a passage in the cranial vertebra extending into the disc using an angled anterior approach that enters the cranial vertebra and terminates at a posterior region of the disc that includes the herniated nucleus pulposus;
placing a disc reconstruction device in the passage, wherein the disc reconstruction device has an expandable implant and an elongate member extending proximally from the expandable implant; and
placing the elongate threaded member at least partially fills the passage in the vertebra.

149-159. (canceled)
